(19) United States
(12) Patent Application Publication
(10) Pub. No.: US 2013/0296250 A1
(43) Pub. Date: Nov. 7, 2013

(54) METHOD FOR TREATMENT OF PSORIASIS

(71) Applicant: HEALOR LTD., (US)

(72) Inventors: Liora BRAIMAN-WIKSMAN, Rishon Le-Zion (IL); Tamar TENNENBAUM, Jerusalem (IL); Yuval SAGIV, Gedera (IL); Marina GARTSBEIN, Petach Tikva (IL); Ephraim BRENER, Rishon Le Zion (IL); Moshe BEN-HAMO, Bene Braq (IL); Liat HAMMER, Modiin (IL)

(21) Appl. No.: 13/742,894

(22) Filed: Jan. 16, 2013

Related U.S. Application Data
(63) Continuation of application No. 13/508,610, now abandoned.

(51) Int. Cl. C07K 7/06

(52) U.S. Cl. CPC C07K 7/06
USPC 514/18.7

(57) ABSTRACT

The present disclosure provides a method and kit for treatment of psoriasis using PKC-alpha inhibitors. Exemplary inhibitors include peptide PKC-alpha inhibitors which specifically inhibit PKC-alpha activity leading to the attenuation and treatment of psoriasis.

### CROSS REFERENCE TO RELATED APPLICATIONS

This application claims the benefit of priority under 35 U.S.C. §119(e) of U.S. Ser. No. 61/405,509, filed Oct. 21, 2010, and U.S. Ser. No. 61/293,794, filed Jan. 11, 2010, the entire content of which are incorporated herein by reference in their entirety.

### BACKGROUND OF THE DISCLOSURE

1. Field of the Disclosure

The disclosure relates generally to methods of treating disease and more specifically to treatment of psoriasis.

2. Background Information

There are two main hypotheses about the basic pathology leading to psoriasis development. The first considers psoriasis as primarily a disorder of excessive growth and reproduction of skin cells. The second hypothesis considers psoriasis as an immune-mediated disorder in which the excessive reproduction of skin cells is secondary to factors produced by the immune system. Accordingly, most drugs for psoriasis target one component of the disease, either the hyper-proliferative state of skin cells, or the skin inflammatory response as presented in psoriasis plaques.

Recent data support the notion that both pathways underlie the pathology of the diseases through a cross talk between skin cells and immunological milieu. Classic genome wide linkage analysis has identified nine locations (loci) on different chromosomes associated with tendency to develop psoriasis named psoriasis susceptibility 1 through 9 (PSORS1 through PSORS9) loci. In these locations several genes were characterized and found to encode for proteins expressed in epidermal cells such as corneodesmosin, expressed in the granular and cornifled layers of the epidermis and upregulated in psoriasis. On the other hand, other psoriasis linked genes encode for proteins involved in modulation of the immune system where characterized such as IL12B on chromosome 5q which expresses interleukin-12B (Frank et at (2009) N Engl J Med 361:496-509).

In addition to genetic predisposition, several in vivo studies have shown the involvement of T helper (Th) 17 cells as well as secretion of cytokines such as interleukins and TNFα, by skin associated cells such as keratinocytes, dendritic and T helper cells, as key players in the development of the inflammatory response involved in the pathogenesis of psoriasis and other autoimmune inflammatory diseases. As used herein, in vivo (Latin for “within the living”) is experimentation using a whole, living organism as opposed to a partial or dead organism, or an in vitro (“within the glass”, for instance, in a test tube or petri dish) controlled environment. The secretion of cytokines such TNFα and Interleukin (IL)-23, which stimulates survival and proliferation of Th17 cells, also serves as a key master cytokine regulator for these diseases. (Fitch et al. (2007) Curr Rheumatol Rep. 9:461-7). Th17 cells within dermis in turn, induce secretion of IL-17A and IL-22. IL-22, in particular, derive keratinocyte hyperproliferation and augment the inflammatory response in psoriasis (Fitch et al. (2007) Curr Rheumatol Rep 9:461-7).

The protein kinase C (PKC) family represents a group of phospholipid dependent enzymes catalyzing the covalent transfer of phosphate from ATP to serine and threonine residues of proteins. The PKC family consists of at least ten members, usually divided into three subgroups: classical, novel and atypical PKCs (FIG. 1). The specific cofactor requirements, tissue distribution, and cellular compartmentalization suggest differential functions and the tuning of specific signaling cascades for each isoform. Thus, specific stimuli can lead to differential responses via isoform specific PKC signaling regulated by their factors, such as: expression, localization, and/or phosphorylation status in particular biological settings. PKC isoforms are activated by a variety of extracellular signals and, in turn, modify the activities of cellular proteins including receptors, enzymes, cytoskeletal proteins, and transcription factors. Accordingly, the PKC family plays a central role in cellular signal processing including regulation of cell proliferation, differentiation, survival and death.

PKCα, which is highly abundant in skin, is the major conventional, Ca²⁺ responsive, PKC isoform in epidermis and it was initially the only cPKC detected in the keratinocytes in vitro and in vivo (Dlugosz et al. (1992) Biomed Pharmacother 46:304; Wang et al. (1993) J Cancer Res Clin Oncol 119:279-287). Therefore, PKCα had been proposed as a key player in Ca²⁺ induced differentiation (Denning et al. (1995) Cell Growth Differ 6:149-157; Dlugosz et al. (1992) Biomed Pharmacother 46:304). Being in epidermis and mainly restricted to suprabasal layers (Denning et al. (2004) Int J Biochem Cell Biol 36:1141-1146), PKCα is involved in cell cycle withdrawal and primarily associated with the keratin cytoskeleton and desmosomal cell—-cell junctions (Jansen et al. (2001) Int J Cancer 93:635-643; Tibudan et al. (2002) J Invest Dermatol. 119:1282-1289). Since, upon exposure to the classical PKC activator TPA (12-O-tetradecanoylphorbol-13-acetate), spinous markers were suppressed, PKCα was thought to be largely responsible for the shift from spinous to granular differentiation as a result of TPA activation (Dlugosz and Yuspa (1993) J Cell Biol 120:217-225; Lee et al. (1998) J Invest Dermatol 111:762-766; Matsui et al. (1992) J Invest Dermatol 99:565-571; Punnonen et al. (1993) J Invest Dermatol 101:719-726). Indeed, blocking PKCα activity or its synthesis by antisense oligonucleotides appeared to abolish granular markers and revive spinous markers like K1 and K10. Likewise, implementation of dominant negative PKCα appeared to restore the (late) spinous marker involucrin (Deucher et al. (2002) Biol Chem 277:17032-17040). Accordingly, defective differentiation in skin cancer (Tennenbaum et al. (1993) Cancer Res 3:4803-4810; Tomakidi et al. (2003) J Pathol 200:298-307) correlates with elevated PKCα activity, also observed in tumor cells in vitro (Dlugosz et al. (1992) Biomed Pharmacother 46:304; Yang et al. (2003) J Cell Physiol. 195:249-259). However, overexpression of PKCα in normal human keratinocytes did not appear to alter their differentiation pattern (Deucher et al. (2002) J Biol Chem 277:17032-17040). The influence of PKCα on the cellular traffic and membrane recruitment of β1-integrin during migration (Ng et al. (1999) EMBO J 18:3909-3923) may well promote both wound reepithelialization and tumor cell invasion.

Overexpression of PKCα in transgenic mice has appeared to induce a striking inflammatory response, increased epidermal thickening and edema correlated with neutrophil infiltration, multiple micro-abscesses, and a marked increase of inflammatory cytokines and chemokines, such as TNFα, MIP-2, COX-2 or macrophage inflammatory protein (MIP). These results implicate PKCα in the epidermal inflammatory response (Wang and Smart (1999) J Cell Sci 112:3497-3506). Treatment with TPA (a PKCα activator) apparently caused epidermal hyperplasia, intra-epidermal inflammation, and massive apoptosis (Cataisson et al. (2003) J Immunol 171:2703-2713; Jansen et al. (2001) Int J Cancer 93:635-643). In addition, recent in vivo studies in PKC isoenzyme-selective knockout and transgenic mice appear to have highlighted distinct functions of individual PKCs in the immune system. These genetic analyses, along with biochemical studies appear to indicate that PKC-regulated signaling pathways play a significant role in many aspects of the immune responses. For example, members of the PKC family appear crucial in T cell signaling pathways. Particularly, PKCα, isotype appears to determine the nature of lymphocyte-specific in vivo effector. PKCα is also discussed as being involved in macrophages activation and was apparently shown to be involved in mast cell signaling (Cataisson et al. (2005) J Immunol 174:1686-1692). Therefore, PKC isotypes are validated drug targets in adaptive immunity.

Current therapy for psoriasis include options which involve drugs that slow the rapid proliferation of skin cells and help reduce scaling on one hand (such as Vitamin D), and drugs that are aimed to reduce inflammation (mainly steroids) or suppress components of the immune system on the other hand. The majority of drugs available today target basically a single component of the disease, either by blocking keratinocytes proliferation, or by suppressing the immune response in order to block inflammation. Consequently, there appear to be no current treatments which result in an effective multi-component approach for the treatment of psoriasis. Such a multi-component treatment would be expected to be more effective than existing single-component solutions.

Currently there appear to be no available treatments of psoriasis which result in simultaneous targeting of multiple components of the pathogenesis of the disease. In addition, while psoriasis is considered a topical chronic skin disease, many of the existing effective drugs are systemic ones, which are based on immune suppression and as a result appear to lead to adverse effects, of which some can be severe. On the other hand, current topical treatments to psoriasis appear to be only moderately effective in reducing symptoms and overcoming pathology. This situation leads to the apparent practice that psoriasis patients commonly visit multiple doctors in a short period of time, indicating their dissatisfaction with available care. As a result, there is a strong need for an effective therapeutic which targets multiple components of the disease's pathogenesis, while retaining a low level of side effects.

### SUMMARY OF THE DISCLOSURE

The present disclosure relates to treatment of psoriasis by administering to a subject an inhibitor of PKCα. Accordingly, in one aspect, the present disclosure provides a method of treating psoriasis in a subject. The method includes administering to the subject an inhibitor of PKCα, thereby treating psoriasis in the subject. In various embodiments, the inhibitor of PKCα is a peptide. In some embodiments, the peptide includes an amino acid sequence selected from SEQ ID NOs: 1-5 and may further include an N-terminal modification, C-terminal modification, or combination thereof. In exemplary embodiments, the peptide is selected from SEQ ID NOs: 6-13.

In another aspect, the present disclosure provides a kit for treating psoriasis in a subject. In various embodiments, the kit includes an inhibitor of PKCα and instructions for administering the inhibitor to the subject. In various embodiments, the inhibitor of PKCα is a peptide. In some embodiments, the peptide includes an amino acid sequence selected from SEQ ID NOs: 1-5 and may further include an N-terminal modification, C-terminal modification, or combination thereof. In exemplary embodiments, the peptide is selected from SEQ ID NOs: 6-13.

### BRIEF DESCRIPTION OF THE DRAWINGS

FIG. 1 is a pictorial representation depicting various members of the PKC family of isoforms.

FIG. 2 is a series of pictorial representations depicting inhibition of PKCα which regulates keratinocytes structure integrity characteristic to psoriasis. Skin tissues were paraffin embedded and stained for hematoxiline and eosine (H&E) general histological staining or distinct markers for the various skin layers including Keratin 14 (K14) for basal layer, Keratin 1 (K1) for spinous layer, Keratin 6 (K6) for keratinocytes migration and PCNA for keratinocytes proliferation. The results demonstrate normalization of skin properties following PKCα inhibition (left column is WT, right column is PKCα knock out).

FIG. 3 is a histogram comparing severity of scaling in different knock out mice as compared to control after treatment with IMQ.

FIGS. 4A, 4B and 4C are a series of pictorial representations showing scaling in knock out mice as compared with control after treatment with IMQ.

FIGS. 5A, 5B and 5C are a series of pictorial representations showing expression of Filaggrin (Fil), Loricrin (Lor) and Keratin 1 (K1).

FIGS. 6A-B are a series of pictorial and graphical representations assessing keratinocytes proliferation in vitro and in vivo. FIG. 6A is a pictorial representation showing expression of PCNA. FIG. 6B is a histogram comparing the percentage of PCNA positive cells treated with HO/02/10 and control.

FIGS. 7A and 7B are a series of pictorial representations showing expression of Filaggrin (Fil), Loricrin (Lor), Keratin 1 (K1, PCNA and Keratin 14 (K14).

FIG. 8 is graphical representation presenting a summary of protein expression data in keratinocytes for various peptide PKCα inhibitors.

FIG. 9 is a histogram comparing the bursting pressure of skin samples treated with HO/02/10 and control.

FIG. 10 is a histogram comparing the anti-inflammatory effect of HO/02/10 on skin wound in B57BL/6J mice after 4 and 9 days post wounds.

FIG. 11 is a histogram comparing cytokine secretion in splenocytes treated with HO/02/10.

FIGS. 12A-12F are a series of pictorial representations showing ICAM expression in basal keratinocytes and endothelial cells in blood vessels of the skin.

FIGS. 13A-13D are is a series of pictorial representations showing ICAM expression in basal keratinocytes and endothelial cells in blood vessels of the skin.

FIG. 14 is a histogram comparing the percent of mice exhibiting positive ICAM-1 staining at wound edges.

FIG. 15 is a histogram comparing the number of cells per field of Iba-1 positively stained cells.

FIGS. 16A-16C are a series of pictorial and graphical representations showing MAC-2 expression in keratinocytes. FIGS. 16A-16B are a series of stains showing MAC-2 expression. FIG. 16C is a histogram comparing the number of cells per field of MAC-2 positively stained cells with control, 1, 10 and 100 micrograms per mL PKCα inhibitor (from left).

FIGS. 17A-D are a series of histograms comparing cytokine secretion in LPS activated keratinocytes treated with HO/02/10. FIG. 17A compares secretion of IL-6, IL-1α, and GM-CSF. FIG. 17B compares secretion of G-CSF. FIG. 17C compares secretion of MIP-2. FIG. 17D compares secretion of KC.

FIGS. 18A-C are a series of histograms comparing cytokine secretion in LPS activated macrophages treated with HO/02/10. FIG. 18A compares secretion of G-CSF, KC and MIP-2. FIG. 18B compares secretion of IL1α (left bars of histogram pairs) and TNFα (right bars of histogram pairs). FIG. 18C compares secretion of IL1β (left bars of histogram pairs) and IL12 (right bars of histogram pairs).

FIG. 19 is a histogram comparing cytokine secretion in LPS activated keratinocytes treated with peptide PKCα inhibitors.

FIG. 20 is a histogram comparing cytokine secretion in LPS activated keratinocytes treated with peptide PKCα inhibitors.

FIGS. 21A-B are histograms comparing cytokine secretion in TNFα activated keratinocytes treated with peptide PKCα inhibitors. FIG. 21A compares secretion of IL-1A. FIG. 21B compares secretion of IL-6.

FIGS. 22A-B are histograms comparing cytokine secretion in TNFα activated keratinocytes treated with peptide PKCα inhibitors. FIG. 22A compares secretion of G-CSF. FIG. 22B compares secretion of GM-CSF.

FIGS. 23A-B are histograms comparing cytokine secretion in TNFα activated keratinocytes treated with peptide PKCα inhibitors. FIG. 23A compares secretion of MIP-2. FIG. 22B compares secretion of IP-10.

FIGS. 24A-B are histograms comparing cytokine secretion in IL-17A activated keratinocytes treated with peptide PKCα inhibitors. FIG. 24A compares secretion of IL-1A. FIG. 24B compares secretion of IL-6.

FIGS. 25A-B are histograms comparing cytokine secretion in IL-17A activated keratinocytes treated with peptide PKCα inhibitors. FIG. 25A compares secretion of TNFα. FIG. 25B compares secretion of IP-10.

FIGS. 26A-B are histograms comparing cytokine secretion in IL-17A activated keratinocytes treated with peptide PKCα inhibitors. FIG. 26A compares secretion of G-CSF. FIG. 26B compares secretion of GM-CSF.

FIG. 27A-B are histograms comparing cytokine secretion in IL-17A activated keratinocytes treated with peptide PKCα inhibitors. FIG. 27A compares secretion of KC. FIG. 27B compares secretion of MIP-2.

a FIGS. 28A-28E are a series of pictorial and graphical representations showing down regulation of T cell infiltration to the dermis and epidermis during the inflammatory stage after treatment with HO/02/10. FIGS. 8A-28D are a series of stains using anti-CD3 antibodies. FIG. 28E is a histogram comparing the number of cells per field of CD3 positively stained cells.

FIGS. 29A-29C are graphical representations presenting a summary of the effects of treatment using the peptide PKCα inhibitor MPDY-1 on different cell types.

FIG. 30 is a graphical representation showing a schema of the overall effect of HO/02/10 on the psoriatic related pathway.

FIGS. 31A-B are a series of pictorial and graphical representations showing down regulation of neutrophil infiltration to the dermis and epidermis during the inflammatory stage after treatment with HO/02/10. FIG. 31A is a stain using neutrophil specific antibodies. FIG. 31B is a histogram comparing the number of cells per field of neutrophil specific positively stained cells.

FIG. 32 is a histogram comparing cytokine secretion in LPS activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 33 is a histogram comparing cytokine secretion in LPS activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 34 is a histogram comparing cytokine secretion in LPS activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 35 is a histogram comparing cytokine secretion in LPS activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 36 is a histogram comparing cytokine secretion in LPS activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 37 is a histogram comparing cytokine secretion in TNFα activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 38 is a histogram comparing cytokine secretion in TNFα activated keratinocytes treated with peptide PKCα inhibitors including MPDY-1 (SEQ ID NO: 6), AWOT-1 (SEQ ID NO. 7), AIP-2 (SEQ ID NO: 8), ATP-1 (SEQ ID NO: 9), and PPDY (SEQ ID NO: 10).

FIG. 39 is a histogram comparing cytokine secretion in TNFα activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 40 is a histogram comparing cytokine secretion in LPS activated keratinocytes treated with peptide PKCα inhibitors including MPDY-1 (SEQ ID NO: 6), AWOT-1 (SEQ ID NO. 7), AIP-2 (SEQ ID NO: 8), AIP-1 (SEQ ID NO: 9), and PPDY (SEQ ID NO: 10).

FIG. 41 is a histogram comparing cytokine secretion in LPS activated keratinocytes treated with peptide PKCα inhibitor MPDY-1 (SEQ ID NO: 6).

FIG. 42 is a histogram comparing cytokine secretion in LPS activated keratinocytes treated with peptide PKCα inhibitor MPDY-1 (SEQ ID NO: 6).

FIG. 43 is a histogram comparing cytokine secretion LPS activated keratinocytes treated with peptide PKCα inhibitor MPDY-1 (SEQ ID NO: 6).

FIG. 44 is a histogram comparing cytokine secretion in LPS activated keratinocytes treated with peptide PKCα inhibitor AWOT-1 (SEQ ID NO: 7).

FIG. 45 is a histogram comparing cytokine secretion in LPS activated keratinocytes treated with peptide PKCα inhibitors MPDY-1 (SEQ ID NO: 6), AWOT-1 (SEQ ID NO: 7), and AIP-2 (SEQ ID NO: 8).

FIG. 46 is a histogram comparing cytokine secretion in LPS activated keratinocytes treated with peptide PKCα inhibitors MPDY-1 (SEQ ID NO: 6), AWOT-1 (SEQ ID NO: 7), and AIP-2 (SEQ ID NO: 8).

FIG. 47 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 48 is a histogram comparing cytokine secretion in TNFα activated keratinocytes treated with peptide PKCα inhibitors MPDY-1 (SEQ ID NO: 6), AWOT-1 (SEQ ID NO: 7), and AIP-2 (SEQ ID NO: 8).

FIG. 49 is a histogram comparing cytokine secretion in TNFα activated keratinocytes treated with peptide PKCα inhibitors MPDY-1 (SEQ ID NO: 6), AWOT-1 (SEQ ID NO: 7), and AIP-2 (SEQ ID NO: 8).

FIG. 50 is a histogram comparing cytokine secretion in TNFα activated keratinocytes treated with peptide PKCα inhibitors MPDY-1 (SEQ ID NO: 6), AWOT-1 (SEQ ID NO: 7), and AIP-2 (SEQ ID NO: 8).

FIG. 51 is a histogram comparing cytokine secretion in TNFα activated keratinocytes treated with peptide PKCα inhibitors MPDY-1 (SEQ ID NO: 6), AWOT-1 (SEQ ID NO: 7), and AIP-2 (SEQ ID NO: 8).

FIG. 52 is a histogram comparing cytokine secretion in TNFα activated keratinocytes treated with peptide PKCα inhibitors MPDY-1 (SEQ ID NO: 6), AWOT-1 (SEQ ID NO: 7), and AIP-2 (SEQ ID NO: 8).

FIG. 53 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 54 is a histogram comparing cytokine secretion in IL-17A activated keratinocytes treated with peptide PKCα inhibitors MPDY-1 (SEQ ID NO: 6), AWOT-T (SEQ ID NO: 7), and AIP-2 (SEQ ID NO: 8).

FIG. 55 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 56 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 57 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 58 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 59 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 60 is a histogram comparing cytokine secretion in IL-17A activated keratinocytes treated with peptide PKCα inhibitors MPDY-1 (SEQ ID NO: 6), AWOT-1 (SEQ ID NO: 7), and AIP-2 (SEQ ID NO: 8).

FIG. 61 is a histogram comparing cytokine secretion in LPS activated keratinocytes treated with peptide PKCα inhibitors MPDY-1 (SEQ ID NO: 6) and PDY-1 (SEQ ID NO: 13).

FIG. 62 is a tabular summary of results for various PKCα inhibitors of cytokine secretion in keratinocytes treated with LPS, TNFα or IL-17A and inhibitor.

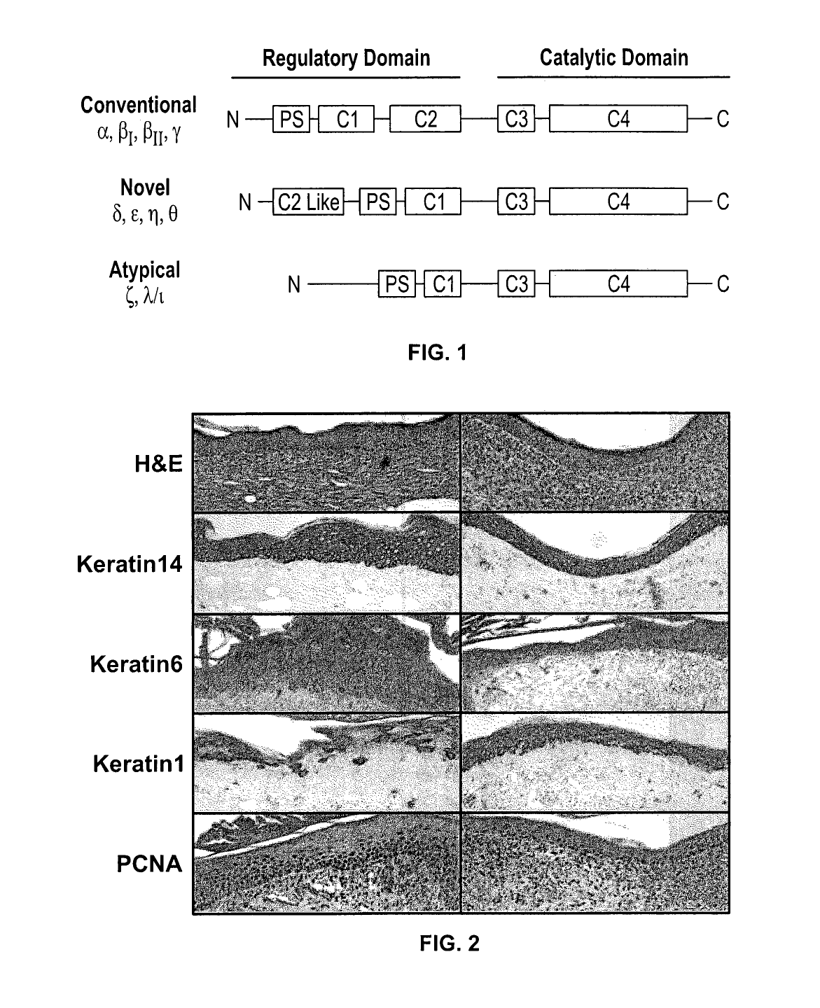

### DETAILED DESCRIPTION OF THE DISCLOSURE

The present disclosure is based on the seminal discovery that inhibitors of PKCα may be administered as an effective treatment for psoriasis. The involvement of PKCα in major cellular processes of skin cells, such as keratinocytes, as well as many components of the immune system, marks it as a potential target for the treatment of skin pathologies. The data presented herein, demonstrate that PKC family isoforms regulate activation processes in skin and immune cells that are associated with psoriasis; see also (Zhao et al. (2008) J Invest Dermatol 128:2190-2197; Cataisson et al. (2003) J Immunol 171:2703-2713).

PKC has been implicated as a factor in patho-physiology of psoriasis (Fisher et a (1993) J Invest Dermatol 101:553-559), apparently being involved in regulating keratinocytes cell death, differentiation, and cutaneous inflammation (Dlugosz and Yuspa (1993) J Cell Biol 120:217-225; Lew et al. (2006) J Korean Med Sci 21:95-99). PKC appears to reduce psoriatic neutrophilic granulocyte accumulation that is ineffective by TNFα antagonist drugs and finally, PKCα over-expression appears to enhance edematous response and increases accumulation of neutrophils in psoriatic epidermis (Wang and Smart (1999) J Cell Sci. 112(Pt 20):3497-506). Specifically, using transgenic mice over-expressing PKCα in the epidermis as a model for psoriasis, it have been shown that keratinocytes produce two types of soluble factors that work independently to recruit neutrophils to the skin (Fitch et al. (2007) Curr Rheumatol Rep 9:461-7). Production of both these soluble factors was apparently controlled by a signaling pathway activated by PKCα. Inhibiting PKCα reduced the recruitment of neutrophils to the skin in mice and reduced the production of neutrophil-attracting soluble factors by keratinocytes from individuals with psoriasis (Cataisson et al. (2005) J Immunol 174:1686-1692). These studies appear to situate the PKCα as a promising therapeutic target for psoriasis treatment, however, the present disclose is the first to present effective inhibitors of PKCα for treatment of psoriasis.

The present disclosure discloses and describes selective inhibitors of PKCα, a PKC isoform from the conventional PKC group, useful for treatment of psoriasis, PKCα inhibition promotes strong attenuation of skin inflammation and regulates basal keratinocytes differentiation and proliferation. This unique combination of effects enables the PKCα inhibitors described herein, and related and similar ones, to halt inflammation while controlling the pace of scaling in psoriatic plaques. Furthermore, in contrast to current anti-inflammatory treatments that include corticosteroids and systemic drugs or various biologics that appear or are reputed to suppress the immune response, the PKCα inhibitors of the present disclosure offer a distinct local therapeutic solution without adverse side effects as well as exhibiting an exemplary safety profile.

It is to be understood that this disclosure is not limited to particular compositions, methods, and experimental conditions described, as such methods and conditions may vary. It is also to be understood that the terminology used herein is for purposes of describing particular embodiments only, and is not intended to be limiting, as the scope of the present disclosure will be limited only in the appended claims.

The principles and operation of the methods according to the present disclosure may be better understood with reference to the figures and accompanying descriptions.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of skill in the art to which this disclosure belongs. Although any methods and materials similar or equivalent to those described herein can be used in the practice or testing of the disclosure, some preferred methods and materials are now described.

As used herein, the term “subject” refers to a mammalian subject. As such, treatment of psoriasis of any animal in the order mammalian is envisioned. Such animals include, but are not limited to horses, cats, dogs, rabbits, mice, goats, sheep, non-human primates and humans. Thus, the method of the present disclosure is contemplated for use in veterinary applications as well as human use.

“Treatment” of a subject herein refers to both therapeutic treatment and prophylactic or preventative measures. Those in need of treatment include those already with psoriasis as well as those in which psoriasis is to be prevented. Hence, the subject may have been diagnosed as having psoriasis or may be predisposed or susceptible to psoriasis.

A “symptom” of psoriasis is any morbid phenomenon or departure from the normal in structure, function, or sensation, experienced by the subject and indicative of psoriasis.

The expression “effective amount” refers to an amount of an inhibitor of PKCα, such as the polypeptides of SEQ ID NOs: 1-13, that is effective for preventing, ameliorating or treating psoriasis. Such an effective amount will generally result in an improvement in the signs, symptoms or other indicators of psoriasis, such as scaling and dry cracked skin such that the clearance of redness and scaling is achieved and the normal appearance of skin as well as pain relief associated with inflammation is achieved.

The present disclosure relates to treatment of psoriasis by administering to a subject an inhibitor of PKCα. PKCα inhibitors of the present disclosure affect multiple components of psoriasis by 1) attenuating the inflammatory process in psoriatic plaques; and 2) controlling epidermal scaling in the plaques. Accordingly, in one aspect, the present disclosure provides a method of treating psoriasis in a subject. The method includes administering to the subject an inhibitor of PKCα, thereby treating psoriasis in the subject.

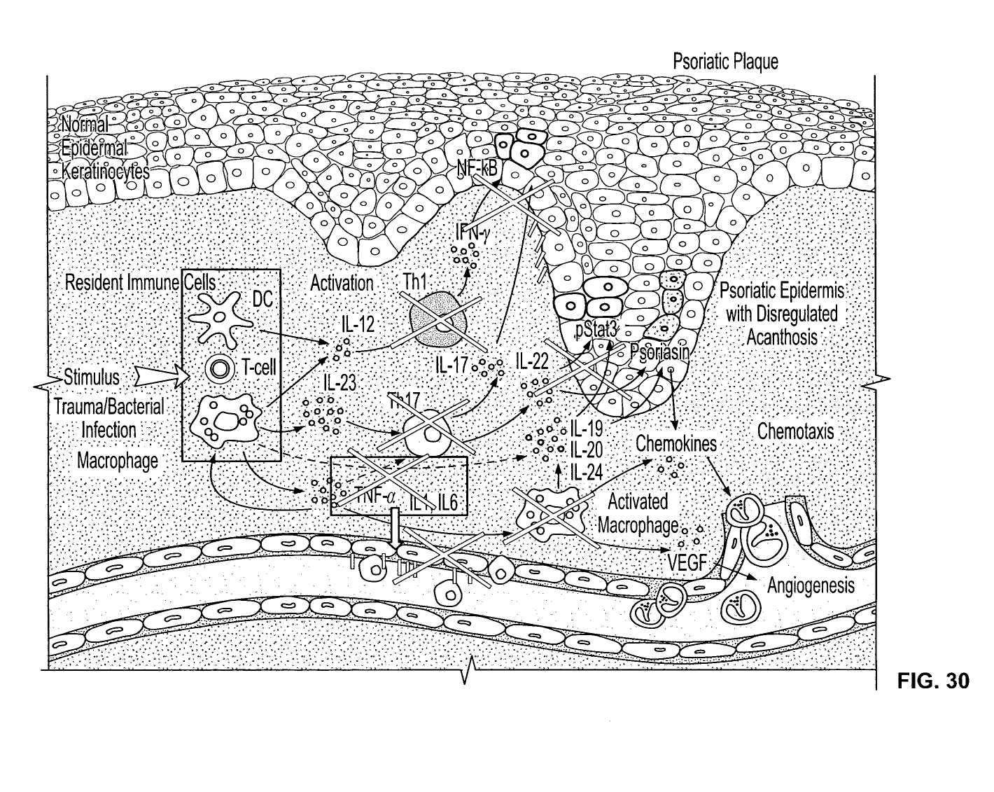

As discussed further in the Examples, the mechanism of action of inhibitors of PCKα has been elucidated implicating their use as an effective therapy for psoriasis. Peptide inhibitors of PCKα have been shown to: 1) normalize epidermal differentiation marker expression by reducing terminal differentiation; 2) attenuate abnormal hyper-proliferation; 3) regulate skin structure and augment skin strength; and/or 4) down-regulate inflammation by differentially affecting different cell type recruitment and activation in various steps of the inflammatory process as summarized, for example, in FIGS. 30A and 30B.

As shown in the Examples, formulations including the PKCα inhibitors of the present disclosure, have been shown to inhibit the secretion of major pro-inflammatory cytokines, such as IL-1, IL-6 and TNFα. Without being bound to a particular theory, it is believed that reducing the level of pro-inflammatory agents prevents the activation of endothelial cells in near-by blood vessels, and thus the recruitment of neutrophiles, macrophages and T cells to the psoriatic plaque. Moreover, TH1 and TH17 cells were shown to be implicated in the pathogenesis of psoriasis by the secretion of specific cytokines, which appear to enhance inflammation or drive keratinocyte hyperproliferation, respectively. The above mentioned pro-inflammatory cytokines appear essential for the development of these TH17 cells (Mangan et al. (2006) Nature 441:231-234; Bettelli et al. (2006) Nature 441:235-238) and for TH1 cell activity. The decrease of their secretion by PKCα inhibitors implicates their use in the effective treatment of psoriasis.

The term “inhibitor” is used herein to describe a molecule that inhibits expression and/or activity of PKCα. Among others, the phosphoryl transfer region, the pseudosubstrate domain, the phorbolester binding sequences, and the phosphorylation sites may be targets for modulation of isoenzyme-specific PKC activity.

The “pseudosubstrate region” or autoinhibitory domain of a PKC isoform is herein defined as a consensus sequence of substrates for the kinase with essentially no phosphorylatable residue. The pseudosubstrate domain is based in the regulatory region, closely resembling the substrate recognition motif, which blocks the recognition site and prevents phosphorylation. Thus, inhibitory peptides of PKCα, such as the polypeptides of the present disclosure, are obtained as by replacing a phosphorylatable residue of serine (S) or tyrosine (T) by alanine (A).

In various embodiments, the inhibitors of PKCα are inhibitors of the pseudosubstrate region of PKCα and are polypeptides. The terms “polypeptide”, “peptide”, or “protein” are used interchangeably herein to designate a linear series of amino acid residues connected one to the other by peptide bonds between the alpha-amino and carboxy groups of adjacent residues.

In general, peptide PKCα inhibitors include the common motif sequence Phe-Ala-Arg-Lys-Gly-Ala (SEQ ID NO: 1). Alternatively, in another embodiment, PKCα inhibitors include the common motif sequence Thr-Leu-Asn-Pro-Gln-Trp-Glu-Ser (SEQ ID NO: 5).

Peptide PKCα inhibitors typically contain between 6 and 12 amino acids, but may be longer or shorter in length. In various embodiment, a PKCα inhibitor may range in length from 6 to 45, 6 to 40, 6 to 35, 6 to 30, 6 to 25, 6 to 20, 6 to 15, or 6 to 10 amino acids. In one embodiment the PKCα inhibitor includes 6, 7, 8, 9, 10, 11, 12, 13, 14 or 15 amino acids.

While the peptide PKCα inhibitors may be defined by motif sequences, one skilled in the art would understand that peptides that have similar sequences may have similar functions. Therefore, peptides having substantially the same sequence or having a sequence that is substantially identical or similar to a PKCα inhibitors including the motif sequences defined by SEQ ID NOs: 1 and 5 are intended to be encompassed. As used herein, the term “substantially the same sequence” includes a peptide including a sequence that has at least 60+% (meaning sixty percent or more), preferably 70+%, more preferably 80+%, and most preferably 90+%, 95+%, or 98+% sequence identity with the motif sequences defined by SEQ ID NOs: 1 and 5 and inhibit PKCα activity.

A further indication that two polypeptides are substantially identical is that one polypeptide is immunologically cross reactive with that of the second. Thus, a polypeptide is typically substantially identical to a second polypeptide, for example, where the t two peptides differ only by conservative substitutions.

The term “conservative substitution” is used in reference to proteins or peptides to reflect amino acid substitutions that do not substantially alter the activity (for example, antimicrobial activity) of the molecule. Typically conservative amino acid substitutions involve substitution of one amino acid for another amino acid with similar chemical properties (for example, charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K) 5) Isoleucine (I), Leucine (L), Methionine (M), Valine (V); and 6) Phenylalanine (F), Tyrosine (Y), and Tryptophan (W).

The term “amino acid” is used in its broadest sense to include naturally occurring amino acids as well as non-naturally occurring amino acids including amino acid analogs. In view of this broad definition, one skilled in the art would know that reference herein to an amino acid includes, for example, naturally occurring proteogenic (L)-amino acids, (D)-amino acids, chemically modified amino acids such as amino acid analogs, naturally occurring non-proteogenic amino acids such as norleucine, and chemically synthesized compounds having properties known in the art to be characteristic of an amino acid. As used herein, the term “proteogenic” indicates that the amino acid can be incorporated into a protein in a cell through a metabolic pathway.

The terms “identical” or percent “identity” in the context of two polypeptide sequences, refer to two or more sequences or sequences or subsequences that are the same or have a specified percentage of amino acid residues that are the same, when compared and aligned for maximum correspondence, as measured using a sequence comparison algorithm or by visual inspection.

The phrase “substantially identical,” in the context of two polypeptides, refers to two or more sequences or subsequences that have at least 60%, preferably 80%, most preferably 90-95% amino acid residue identity, when compared and aligned for maximum correspondence, as measured using a sequence comparison algorithm or by visual inspection.

As is generally known in the art, optimal alignment of sequences for comparison can be conducted, for example, by the local homology algorithm of Smith & Waterman ((1981) Adv Appl Math 2:482), by the homology alignment algorithm of Needleman & Wunsch ((1970) J Mol Biol 48:443), by the search for similarity method of Pearson & Lipman ((1988) Proc Natl Acad Sci USA 85:2444), by computerized implementations of these algorithms, by visual inspection, or other effective methods.

Peptide PKCα inhibitors may have modified amino acid sequences or non-naturally occurring termini modifications. Modifications to the peptide sequence can include, for example, additions, deletions or substitutions of amino acids, provided the peptide produced by such modifications retains PKCα inhibitory activity. Additionally, the peptides can be present in the formulation with free termini or with amino-protected (such as N-protected) and/or carboxy-protected (such as C-protected) termini. Protecting groups include: (a) aromatic urethane-type protecting groups which include benzyloxycarbonyl, 2-chlorobenzyloxycarbonyl, 9-fluorenylmethyloxycarbonyl, isonicotinyloxycarbonyl and 4-methoxybenzyloxycarbonyl; (b) aliphatic urethane-type protecting groups which include t-butoxycarbonyl, t-amyloxycarbonyl, isopropyloxycarbonvy, 2-(4-biphenyl)-2-propyloxycarbonyl, allyloxycarbonyl and methylsulfonylethoxycarbonyl; (c) cycloalkyl urethane-type protecting groups which include adamantyloxycarbonyl, cyclopentyloxycarbonyl, cyclohexyloxycarbonyl and isobornyloxycarbonyl; (d) acyl protecting groups or sulfonyl protecting groups. Additional protecting groups include benzyloxycarbonyl, t-butoxycarbonyl, acetyl, 2-propylpentanoyl, 4-methylpentanoyl, t-butylacetyl, 3-cyclohexylpropionyl, n-butanesulfonyl, benzylsulfonyl, 4-methylbenzenesulfonyl, 2-naphthalenesulfonyl, 3 naphthalenesulfonyl and 1-camphorsulfonyl.

In one embodiment, the PKCα inhibitor is N-acylated, preferably by an acyl group derived from a C12-C20 fatty acid, such as C14 acyl (myristoyl) or C16 acyl (palmitoyl). In an exemplary embodiment, the peptide is an N-myristoylated peptide defined by SEQ ID NO: 6 (herein referred to as MPDY-1), SEQ ID NO: 8, or SEQ ID NO: 12. In another exemplary embodiment, the peptide is an N-palmitylated peptide defined by SEQ ID NO: 10 (herein referred to as PPDY-1) or SEQ ID NO: 11.

Examples of peptide PKCα inhibitors that can be used include, without being limited to, peptides of SEQ ID NOs: 1-5 as shown in Table 1, or the peptides of SEQ ID NOs: 6-13 of Table 1 which are shown having particular modifications or terminal protecting groups.

TABLE 1
PKCα Isoform Inhibitor Peptides

| | SEQ |
|---|---|
| Amino Acid Sequence | ID NO |
| Phe-Ala-Arg-Lys-Gly-Ala | 1 |
| Phe-Ala-Arg-Lys-Gly-Ala-Leu-Arg-Gln | 2 |
| Phe-Ala-Arg-Lys-Gly-Ala-Leu | 3 |
| Phe-Ala-Arg-Lys-Gly-Ala-Arg-Gln | 4 |
| Thr-Leu-Asn-Pro-Gln-Trp-Glu-Ser | 5 |
| Myristoyl-Phe-Ala-Arg-Lys-Gly-Ala-Leu-Arg-Gln-OH | 6 |
| H-Phe-Ala-Arg-Lys-Gly-Ala-Leu-Arg-Gln-OH | 7 |
| Myristoyl-Phe-Ala-Arg-Lys-Gly-Ala-Leu-OH- | 8 |
| trifluoracetate salt | |
| H-Thr-Leu-Asn-Pro-Gln-Trp-Glu-Ser-OH | 9 |
| Palmitoyl-Phe-Ala-Arg-Lys-Gly-Ala-Leu-Arg-Gln-OH- | 10 |
| acetate salt | |
| Palmitoyl-Phe-Ala-Arg-Lys-Gly-Ala-Arg-Gln-OH | 11 |
| Myristoyl-Phe-Ala-Arg-Lys-Gly-Ala-Leu-OH | 12 |
| H-Phe-Ala-Arg-Lys-Gly-Ala-Leu-Arg-Gln-OH-acetate salt | 13 |

In various embodiments, PKCα inhibitors may be administered by any suitable means, including topical, parenteral, subcutaneous, intravenous, intraperitoneal, intrapulmonary, intranasal, and/or intralesional administration in order to treat the subject. However, in exemplary embodiments, the PKCα inhibitors, namely peptide PKCα inhibitors, are formulated for topical application, such as in the form of a liquid, cream, gel, ointment, foam, spray or the like.

Therapeutic formulations of the PKCα inhibitors used in accordance with the present disclosure are prepared, for example, by mixing a PKCα inhibitor having the desired degree of purity with optional pharmaceutically acceptable carriers, excipients and/or stabilizers (see, for example: Remington's Pharmaceutical Sciences, 16th edition, Osol, A. Ed. (1980)). Acceptable carriers, excipients, or stabilizers are expectedly nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

In exemplary embodiments, the PKCα inhibitors, namely peptide PKCα inhibitors, are formulated in a cream. The inhibitors of PKCα are ideal for topical treatment of psoriasis since the activity of PKC enzymes, such as PKCα may be specifically targeted. Inhibition of PKCα is achieved by the ability of the inhibitors to selectively modulate PKCα in lower concentrations, without affecting other PKC isoforms. Unlike systemic treatments that are used to suppress the immune system and slow down skin cell growth or Immunomodulator drugs (biologics), a topical administration with minimal systemic absorption of PKCα inhibitors affects only the area of skin where applied.

An exemplary formulation for topical administration is disclosed in Example 4, in which the peptide MPDY-1 is formulated as a cream for topical administration. However, one skilled in the art would understand that alterations of the formulation may be made while retaining the essential characteristics of the cream, such as viscosity, stabilization, non-toxicity and the like. Also, one skilled in the art would recognize that the formulation may be used as a vehicle for any of the peptide PKCα inhibitors of the present disclosure.

In another embodiment, an article of manufacture, such as a kit containing materials useful for the treatment of psoriasis as described above is provided. In various embodiments, the kit includes an inhibitor of PKCα, namely a peptide PKCα inhibitor as disclosed herein, and instructions for administering the inhibitor to the subject.

The term “instructions” or “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, and the like.

As disclosed herein, the inhibitor of PKCα may be formulated for a specific route of administration. As such, the kit may include a formulation including an inhibitor of PKCα that is contained in a suitable container, such as, for example, tubes, bottles, vials, syringes, and the like. The containers may be formed from a variety of materials such as glass or plastic. The container holds or contains a composition that is effective for treating the psoriasis and may have a sterile access port (for example the container may be an intravenous solution bag or a vial having a stopper pierceable by a hypodermic injection needle). At least one component in the formulation is an inhibitor of PKCα. The label or package insert indicates that the composition is used for treating psoriasis in a subject suffering therefrom with specific guidance regarding dosing amounts and intervals for providing the formulation including an inhibitor of PKCα. The article of manufacture may further include other materials desirable from a commercial and user standpoint, including other buffers, diluents, filters, needles, and syringes.

It will be understood, that the specific dose level and frequency of dosage for any particular subject in need of treatment may be varied and will depend upon a variety of factors including the activity of the inhibitor of PKCα employed, the metabolic stability and length of action of that compound, the age, body weight, general health, sex, diet, mode and time of administration, the severity of the particular condition, and the host undergoing therapy. Generally however, dosage will approximate that which is typical for known methods of administration of the specific inhibitor of PKCα. Persons of skill in the art can easily determine optimum dosages, dosing methodologies and repetition rates. The exact formulation and dosage can be chosen by the individual physician in view of the patient's condition (Fingl et al. “The Pharmacological Basis of Therapeutics”, Ch. 1 p. 1 (1975)).

Thus, depending on the severity and responsiveness of the psoriasis condition to be treated, dosing can be a single or repetitive administration, with course of treatment lasting from several days to several weeks or until cure is effected or diminution of the disorder is achieved.

In various embodiments where the PKCα inhibitor is a peptide, the peptide is provided in the composition at a concentration of between 0.001 and 100 μg/ml. For example, the concentration may be between 0.001 and 100, 0.01 and 50, 0.01 and 10, 0.01 and 1, and 0.01 and 0.5 μg/ml.

In one dosing protocol, the method comprises administering a peptide PKCα inhibitor to the subject topically, for example as a cream. The peptide is topically applied at a concentration of from about 1 μg/ml to about 1000 μg/ml, 1 μg/ml to about 500 μg/ml, 1 μg/ml to about 100 μg/ml, 1 μg/ml to about 10 μg/ml, or 10 μg/ml to about 100 μg/ml. The peptide is administered at least once daily until the condition is treated.

The following examples are provided to further illustrate the embodiments of the present disclosure, but are not intended to limit the scope. While they are typical of those that might be used, other procedures, methodologies, or techniques known to those skilled in the art may alternatively be used.

### Example 1

### Inhibition of PKCα Regulates Keratinocyte Structure Integrity Characteristic to Psoriasis

Inhibition of PKCα was shown to regulate keratinocyte structure integrity characteristic to psoriasis. Skin tissues were paraffin embedded and stained for H&E (hematoxiline and eosine) general histological staining or for distinct markers for the various skin layers including Keratin 14 (K14) for basal layer, Keratin 1 (K1) for spinous layer, Keratin 6 (K6) for keratinocytes migration and PCNA for keratinocytes proliferation. The results demonstrate normalization of skin properties following PKCα inhibition (FIG. 2).

### Example 2

### Models for Assessing In Vivo and Ex Vivo Treatment of Psoriasis

Numerous animal models have been previously used to study psoriasis, however, none of these models were sufficient to adequately mimic the human disease pathology characterized by excessive skin production, formation of new blood vessels, and severe immune dysfunction. In general, to be considered as a useful model of psoriasis, the model has to share some histopathology features with psoriasis, exhibit similar pathogenesis and/or disease mechanism, and respond similarly to therapeutic agents for the treatment of the disease Existing models exhibit several characteristics including acanthosis, altered epidermal differentiation, increase in vascularization, and Leukocytic/T cell infiltration. However, among the existing mice models, not many respond to existing drugs and therapies. As such, existing models were used to develop new in-vitro, ex-vivo and in-vivo models to assess psoriasis treatment which were utilized in the following Examples.

### In-Vitro Models

Developed models included cell culture studies using cells lines and primary cultures of skin derived cells as well as immune cells, utilizing constructs and tools to over-express and inactivate STAT3 and PKCα mediated signaling pathways. A vast set of techniques for the study of skin cell proliferation, migration, differentiation, inflammation and signaling were utilized and proved useful in studying the mechanism of psoriasis development and to study the therapeutic effect of PKCα inhibition in psoriasis.

### In-Vivo Models

A PKCα over-expressing and knockout mouse models were used. Over expression of PKCα in keratinocytes using a K5-PKCα transgenic mice, was shown to exhibit severe intra-epidermal neutrophil infiltration and disruption of the epidermis that mimic conditions such as pustular psoriasis. Both PKCα and DN forms of transgenic mice were established which were studied in vivo by sub-dermal application. In addition, PKCα knockout mice are also used to study the effects of PKCα inactivation on skin structure and function.

A STAT3 over-expressing mouse model used. Among the leading mice models for psoriasis, in terms of similarity to human psoriasis, is a transgenic mouse in which Stat3, is over-expressed in epidermal keratinocytes. These mice, develop psoriasiform epidermal acanthosis and have a cutaneous lymphocytic infiltrate that is predominantly CD4+ in the dermis, and CD8+ in the epidermis, all are features that are similar to psoriasis in human.

Wound as a model for skin inflammation and hyperplasia. A screening methodology was developed to detect and quantitatively assess inflammation in skin lesions in a wound setting which allows to follow cutaneous inflammatory response in the different skin compartments and identify agents that affect this response.

### EX-Vivo-Models

Psoriatic skin grafting on Chick Chorioalantoic Membrane (CAM). A technique of psoriatic skin grafting on Chick Chorioalantoic Membrane (CAM) was developed for the purpose of testing ex-vivo treatment applications. While this technique is commonly used for skin tumor studies and angiogenesis experiments, it was adopted and used for psoriasis studies. This original approach allows the application of new drugs directly on human psoriatic skin, thus creating a more clinically relevant study of new drugs for the treatment of psoriasis. Following grafting, psoriatic human skin is utilized to establish efficacy and timing of various treatments in various formulations, analyzed using morphological, histological and biochemical analysis.

### Example 3

### Attenuation of Scaling in PKCα Knock Out Mice

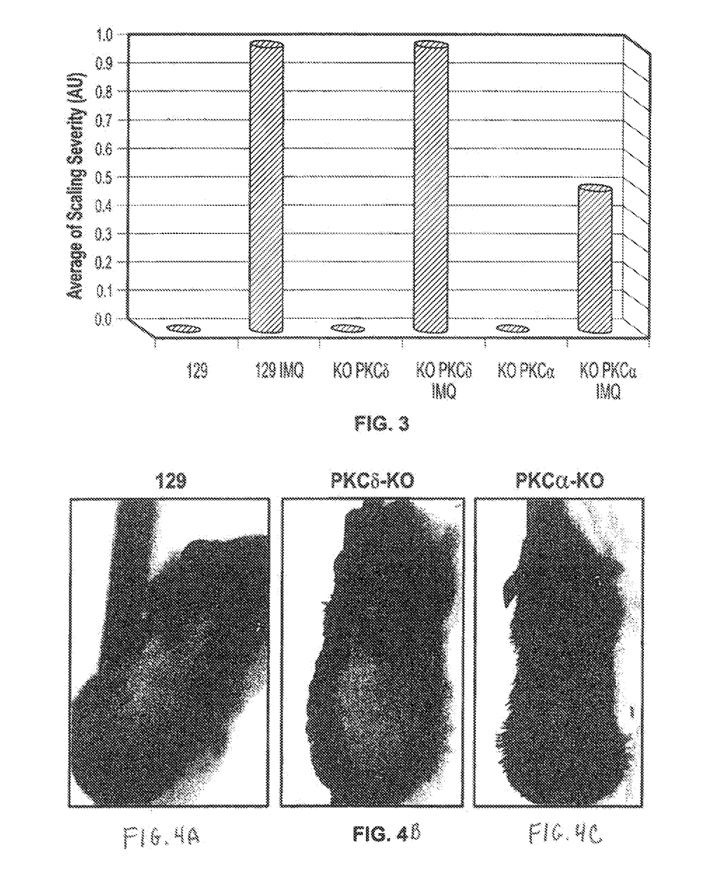

A PKCα knockout mouse model was developed and utilized to study the effects of PKCα inactivation on skin structure and function. As shown in FIGS. 3 and 4, attenuation of scaling was observed in PCKα knock out mice. FIG. 3 is a histogram showing that the average scaling severity was reduced by over 50% in PCKα knock out mice as compared to control evidencing that inhibition of PKCα is a key requirement in treating psoriasis. This is also shown in FIGS. 4A-4C, which is a series of pictures comparing scaling in different mice.

### Example 4

### Topical PKCα Inhibitor Formulation

A topical PKCα inhibitor formulation was developed and assessed for effectiveness in treatment of psoriasis. The peptide PKCα inhibitor MPDY-1 (SEQ ID NO: 6) was formulated in a cream (referred to herein as HO/02/10), the components of which are shown in Table 2.

TABLE 2
MPDY-1 Cream Based Formulation
INGREDIENTS
Water
Glycerine
Propylene Glycol
Methylparaben
Phenoxyethanol
Glyceryl Stearate SE
Cetyl Alcohol
Cosbiol
PEG-40 Stearath
Sucrose Distearate
Isopropyl Myristate
Butylated Hydroxy Toluene
Paraffin Oil
Capric / Caprylic Triglyceride
Vaseline
Propylparaben
MPDY-1

### Example 5

### Effect of PKCα Inhibitors on In Vitro Epidermal Differentiation

The formulation of Example 4 (HO/02/10) was determined to control epidermal differentiation in vitro. Basal keratinocytes differentiate to form the spinous layer, characterized by K1/K10 keratins, the granular layer that is characterized by Loricrin/Filaggrin and the stratum corneum. Defects in expression and incorporation of Loricrin and Filaggrin filaments are associated with various immunological skin diseases including psoriasis. Thus, the effects of HO/02/10, were assessed on skin differentiation and proliferation. As shown in FIGS. 5A-6B, HO/02/10 normalized skin proliferation (PCNA) (FIGS. 6A-6B) and regulated skin differentiation by reducing the expression of Loricrin and Filaggrin, while spinous layer remained unaffected (FIGS. 5A-5C). Since psoriatic skin keratinocytes differentiate rapidly to produce granular and mainly large amounts of corneal cells (scales), while the spinous layer thins, HO/02/10 served to normalize psoriatic skin by amending the skin characteristics toward a normal phenotype.

FIGS. 5A-5C show that HO/02/10 controls epidermal granular differentiation in vitro. Keratinocytes derived from C57BL/6J mice were incubated in medium containing Ca²⁺ to induce keratinocytes differentiation. Cells were then incubated in the presence of HO/02/10 (1 μg/ml). Cells were harvested, run on SDS PAGE gel and immunoblotted using anti-Filaggrin (Fil), anti Loricrin (Lor) and anti-Keratin 1 (K1) antibody.

FIGS. 6A-6B shows that HO/02/10 reduced keratinocytes proliferation in vitro and in vivo. Primary murine keratinocytes from 2 day Balb/c mice were grown for 5 days to reach full confluence in 0.05 mM Ca²⁺ MEM medium. HO/02/10 treatment (10⁻⁶M and 10⁻⁵ M) was applied 6 h prior to induction of differentiation. Cells were harvested, run on SDS PAGE gel and immunoblotted using anti-PCNA antibodies. Results are shown in FIG. 6A. C57Black mice, 8-10 weeks of age were subjected to full thickness wounding in the upper back area to induce epidermis remodeling and differentiation. Following the wounding, mice were treated daily with HO/02/10 (ranged 40-4000 mg/kg/day) for 7 days. At the termination point, mice were euthanized and upper back skin samples were fixed in 4% paraformaldehyde solution, following paraffin embedding and slide preparation. Skin samples were then subjected to immunohistochemical staining utilizing PCNA antibody. (n=18). The results are shown in FIG. 6B.

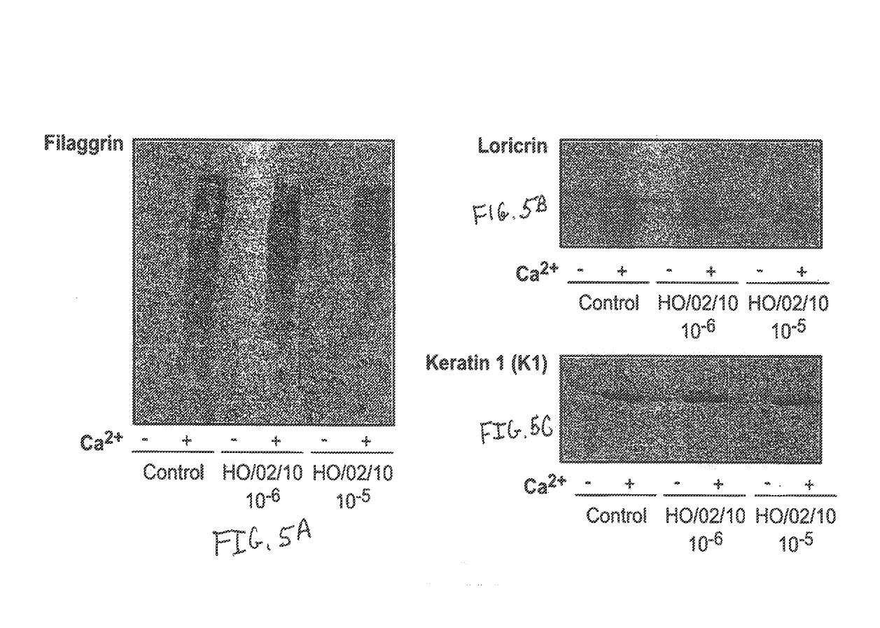

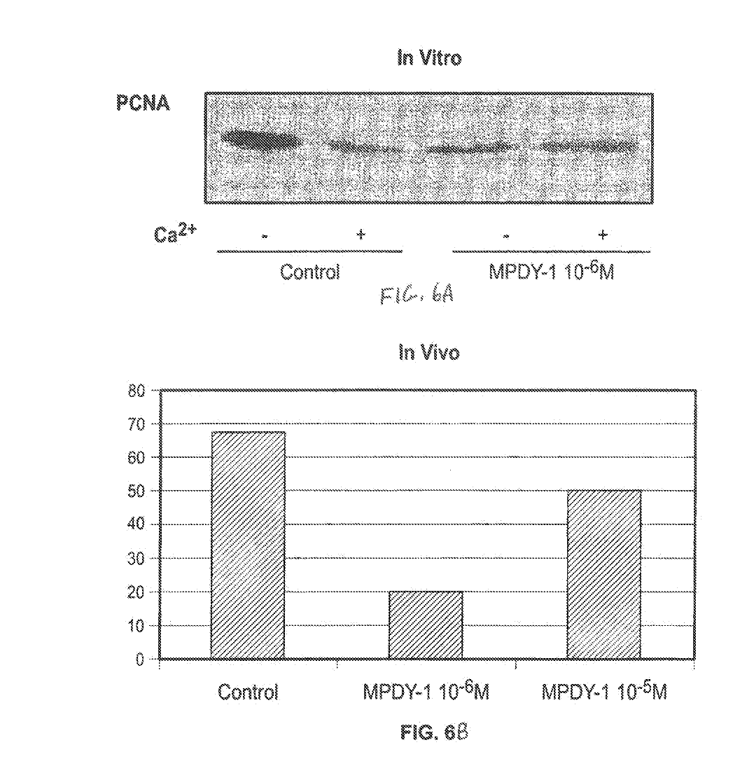

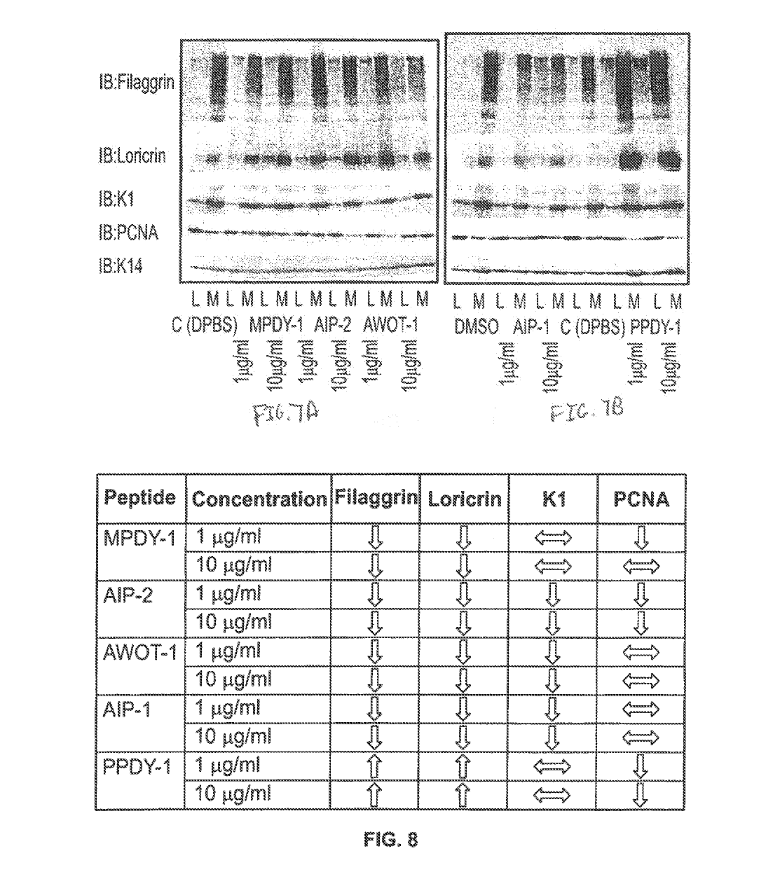

FIGS. 7A, 7B and 8 show additional expression data in keratinocytes utilizing MPDY-1 (SEQ ID NO: 6) as well as data for the peptide PKCα inhibitors AIP-1 (SEQ ID NO: 9), AIP-2 (SEQ ID NO: 8), AWOT-1 (SEQ ID NO: 7) and PPDY-1 (SEQ ID NO: 10). FIG. 7 shows immunohistochemical staining utilizing anti-PCNA, anti-Filaggrin (Fil), anti-Loricrin (Lor), anti-Keratin 1 (K1) and anti-Keratin 14 (K14) antibody in keratinocytes treated with various peptide PKCα inhibitors. FIG. 8 presents a summary of expression data in keratinocytes for various peptide PKCα inhibitors.

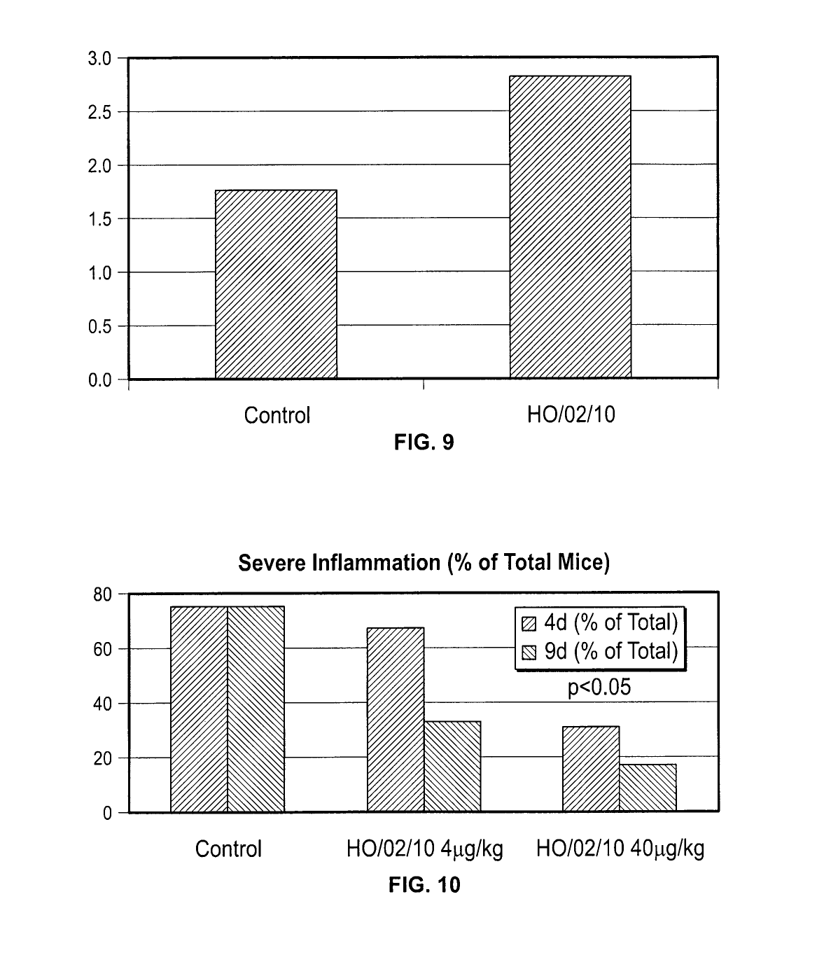

In order to test skin strength and elasticity, a bursting chamber was used to measure the pressure that required for skin samples to burst (a measurable indicator of skin elasticity and durability). The results in FIG. 9, demonstrate that HO/02/10 treated skin exhibited enhanced skin strength. Thus, inhibition of PKCα may be beneficial to psoriatic skin as it was shown to enhance skin integrity and prevent bursting of psoriatic lesions.

FIG. 9 shows that HO/02/10 dramatically enforced skin strength. Mice skin was treated for 14 days with HO/02/10 and subsequently was subjected to bursting pressure analysis. The bursting chamber device consisted of a fixed volume metal cylinder closed on one end and connected to a high-pressure CO² container via a control valve and a manometer. On the other end of the chamber, an adjustable frame was installed in order to mount and hold the tested skin tissue in place. Gas was gradually released into the chamber, and the pressure inside was continuously monitored until bursting of the tested tissue occurs.

### Example 6

### Effect of PKCα a Inhibitors on Skin Inflammation

A methodology was developed to detect and quantitatively assess inflammation in skin lesions in a wound setting which allows one to follow cutaneous inflammatory response in the different skin compartments and identify agents that affect this response (as a preliminary screening). Inflammatory response was considered severe when two of the following three conditions were evident: (1) abscess formation; (2) excessive leukocytosis (>100 cells in a fixed field x200); (3) high WB C/RBC ratio in blood vessels, where >20% of WBC content within the blood vessels is shown in a fixed field x200. Mechanistic characterization of the immunological response is studied utilizing markers to identify infiltration and activation of specific immunological cells. Examples for such markers are: ICAM-1 (as a marker activated basal keratinocytes and endothelial cells), MAC-2 (as a marker for activated macrophages) and CD3 (T cell marker). Using this quantitative method, it was possible to demonstrate a strong anti-inflammatory effect of HO/02/10 and other peptide PCKα inhibitors in intact skin and in skin lesions in different cell types and processes in several animal models.

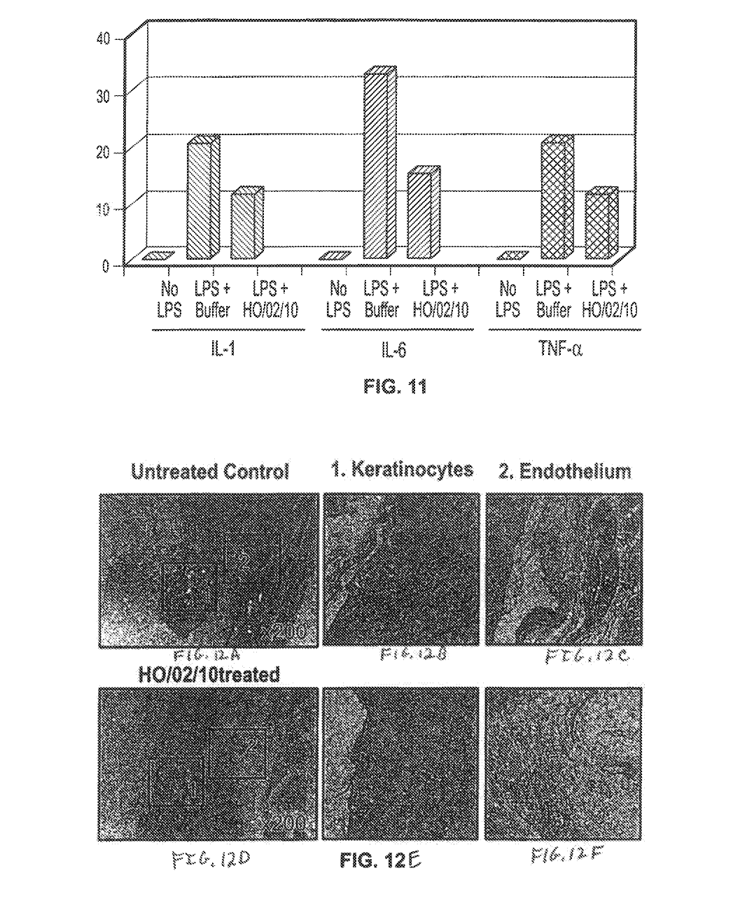

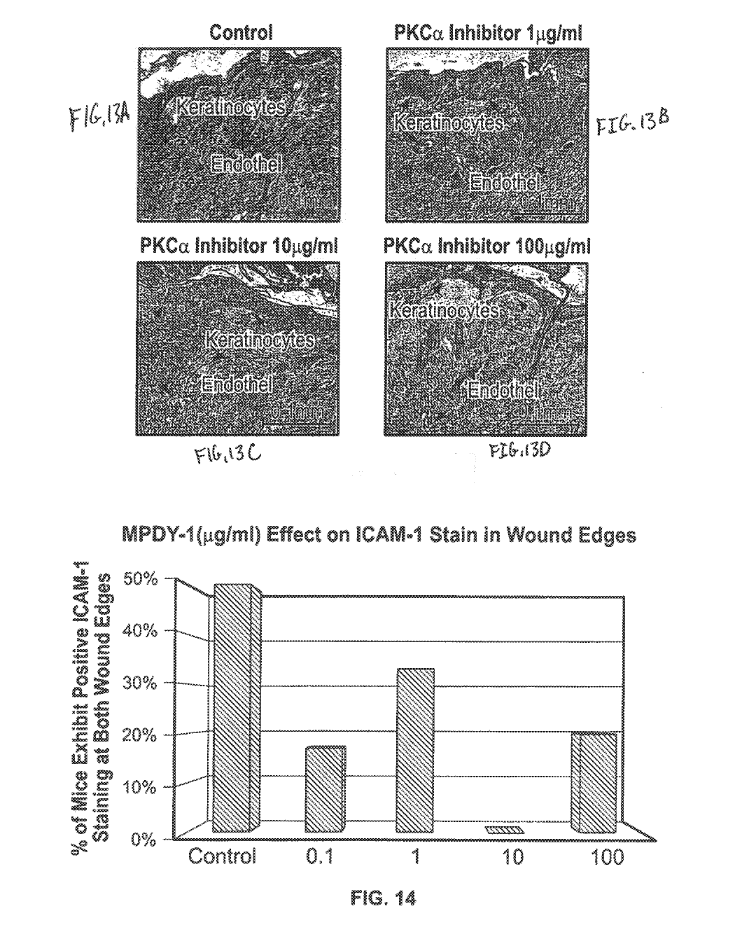

The representative results below demonstrate the anti-inflammatory effect of HO/02/10 on skin wound in B57BL/6J mice after 4 and 9 days post wounds (FIG. 10). FIG. 10 shows the dose response of HO/02/10 effects on inflammation in C57BL/6J mice. Skins of C57BL/6J mice were treated daily by application of HO/02/10 (4 μg/kg/day) or (40 μg/kg/day) (6 mice/group). Treatments were applied topically. Biopsies were collected 4 and 9 days post-wounding. Tissues were excised from euthanized animals for evaluation of inflammation by histology and immunohistochemistry.

HO/02/10 was also shown to decreases pro-inflammatory cytokine secretion from LPS-activated splenocytes. In order to assess general anti inflammatory effects in vitro, mice-derived primary splenocytes were utilized as an immunological model. Splenocytes were derived from C57BL/6J mice, red blood cells were lysed and cells were incubated at 500,000 per well in a 96 well plate. LPS was added (1 μg/ml for IL-1 and TNFα test, and 0.2 ng/ml for IL-6 test), and cells were treated with MPDY-1 (1 μg/ml) or PBS. No LPS was added in negative control samples. Medium was collected after 2 days and the amount of secreted cytokines was quantified using ELISA.

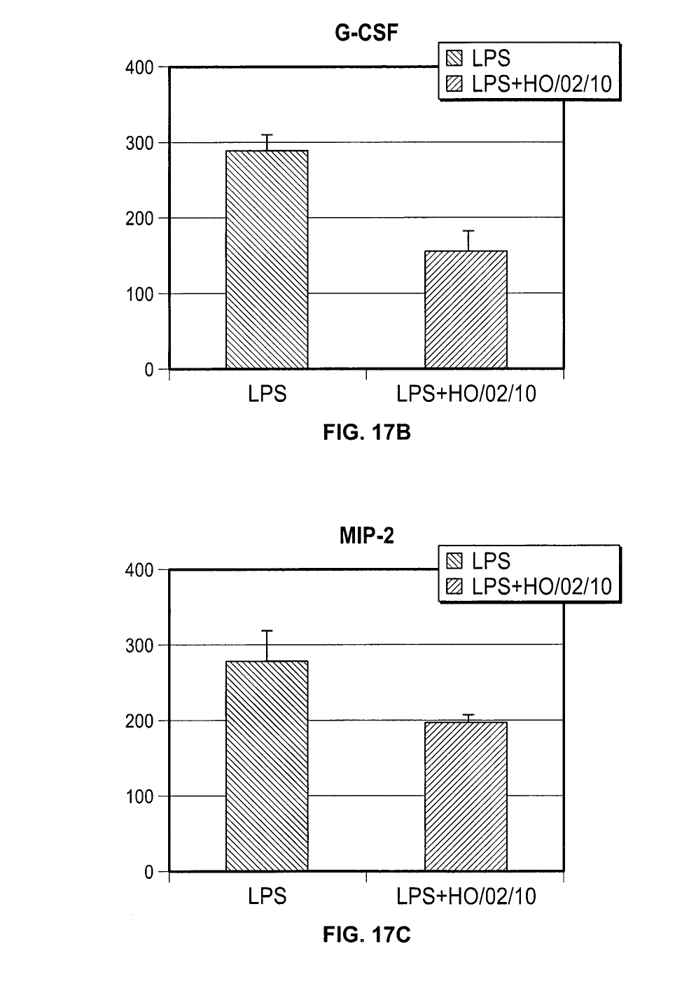

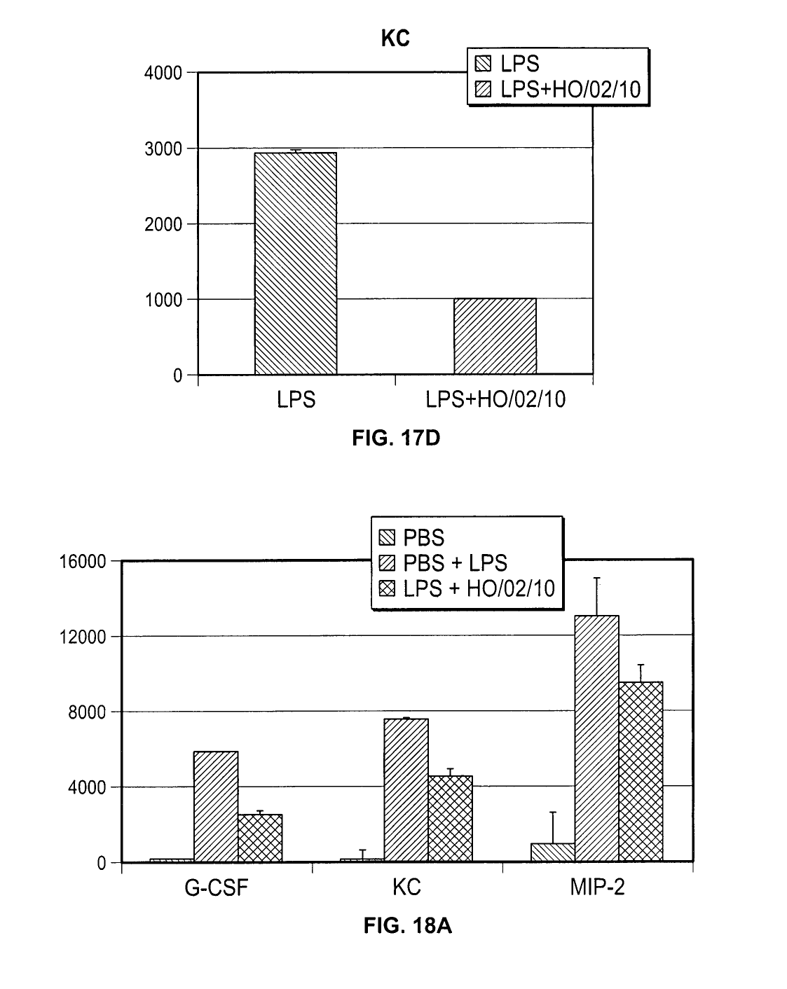

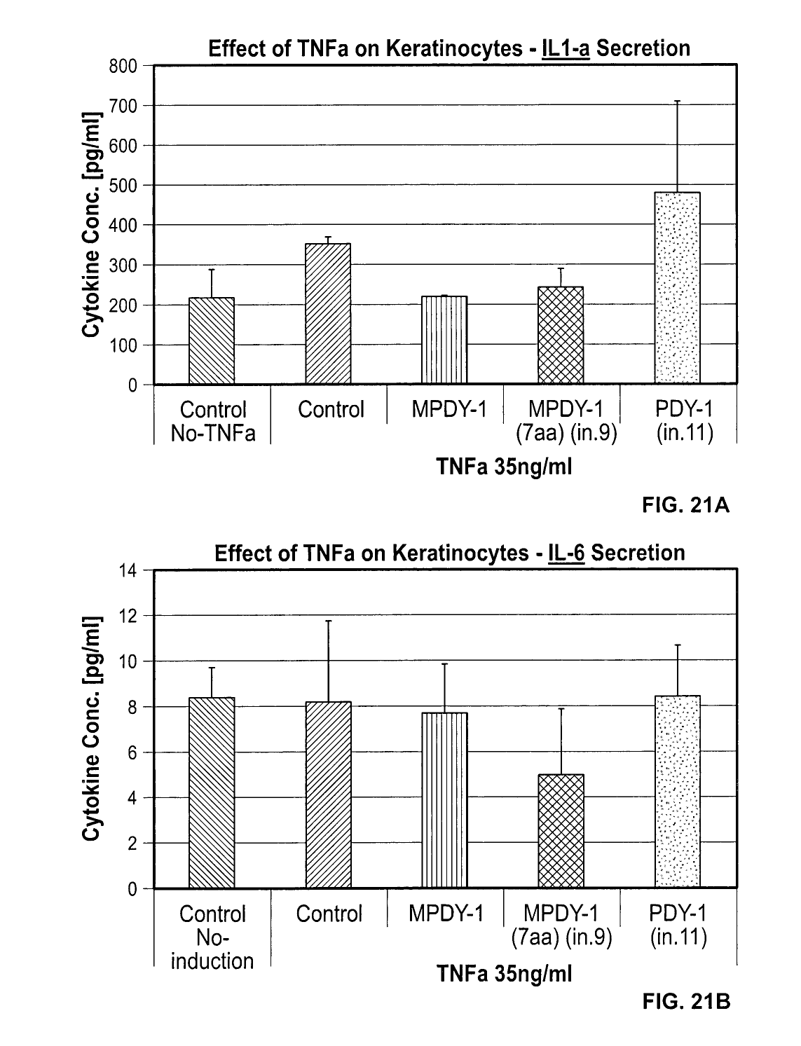

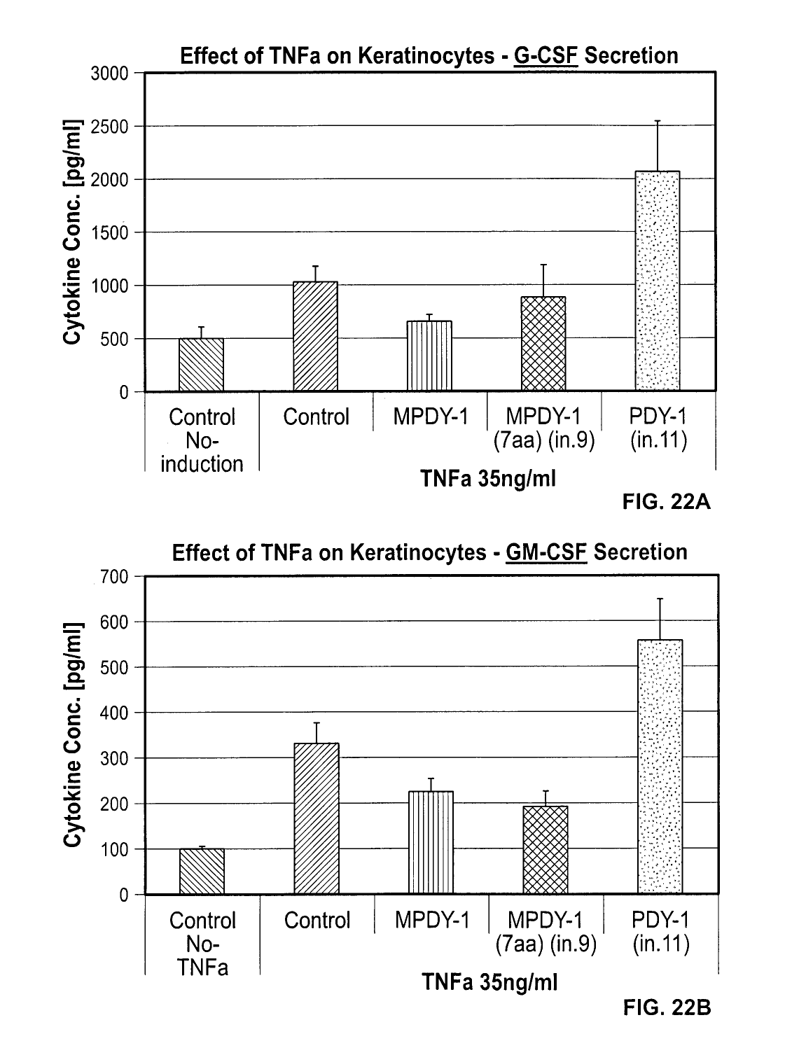

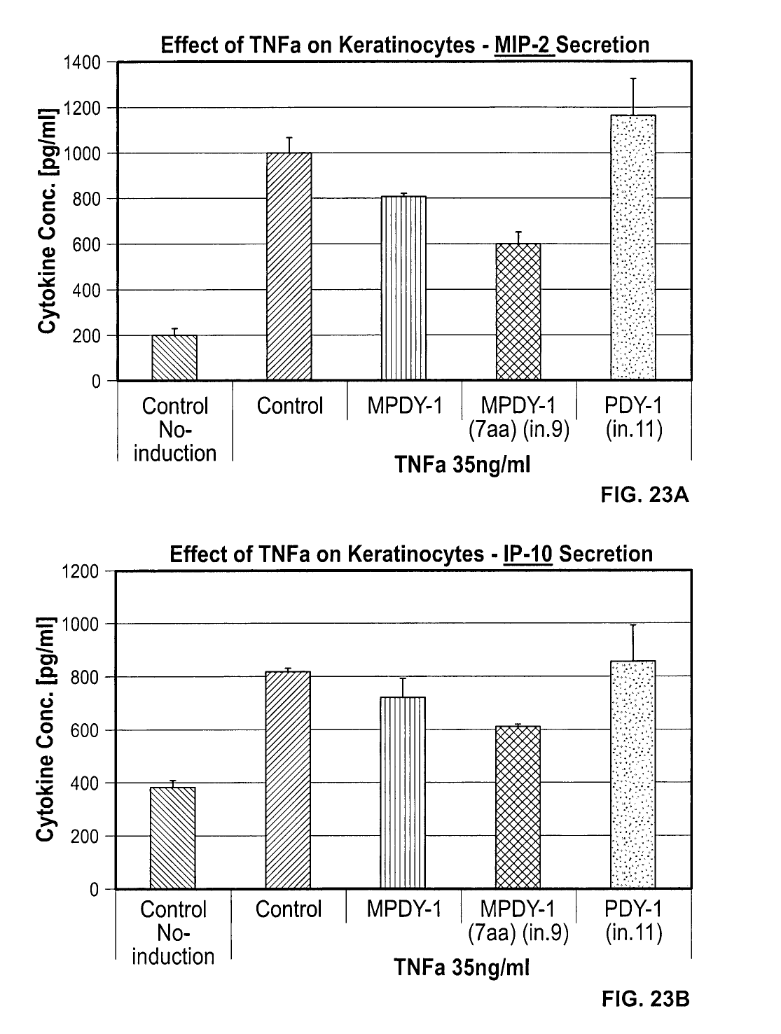

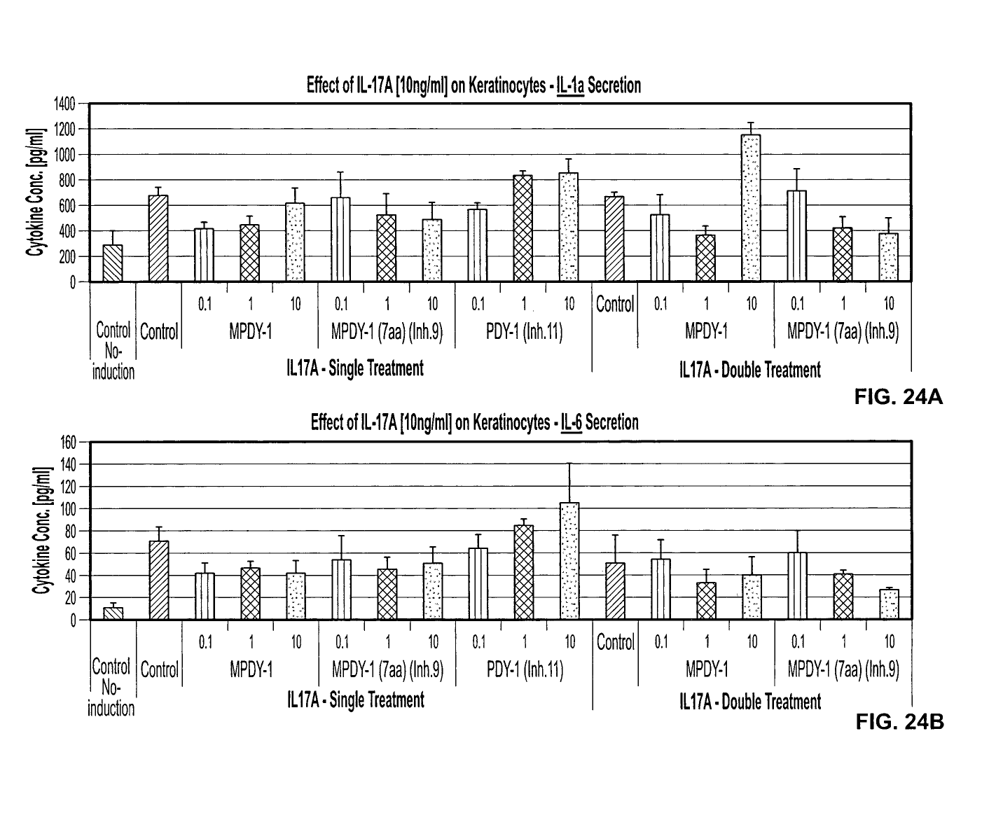

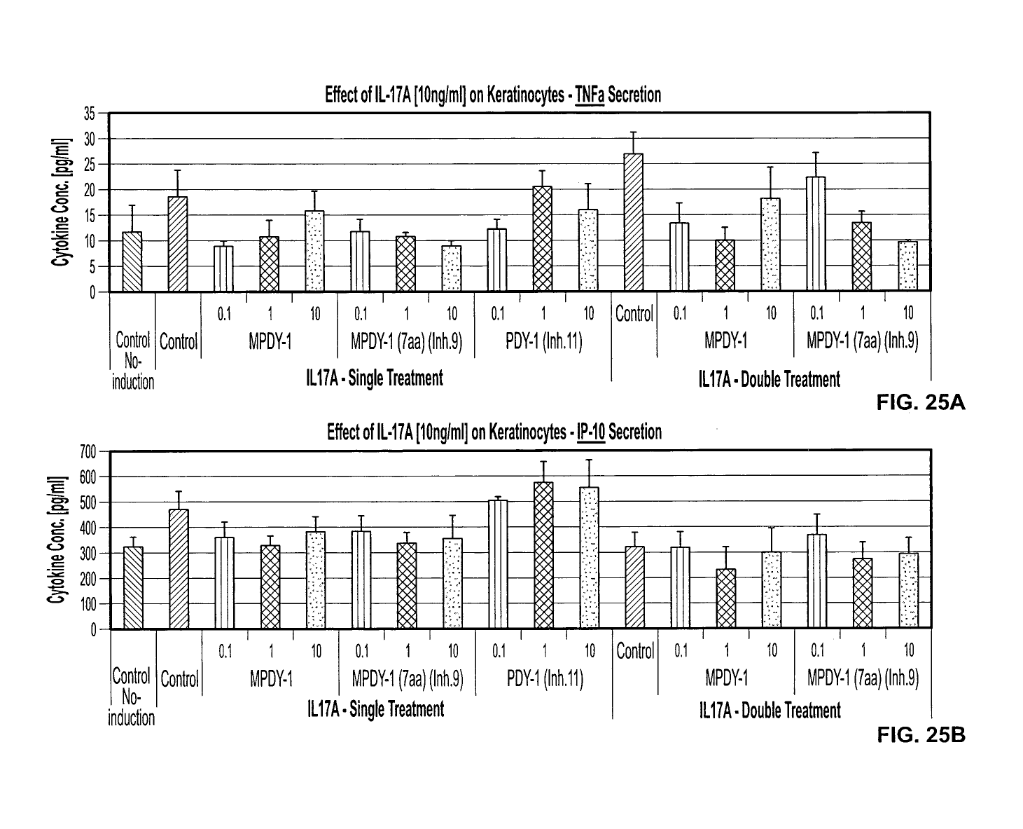

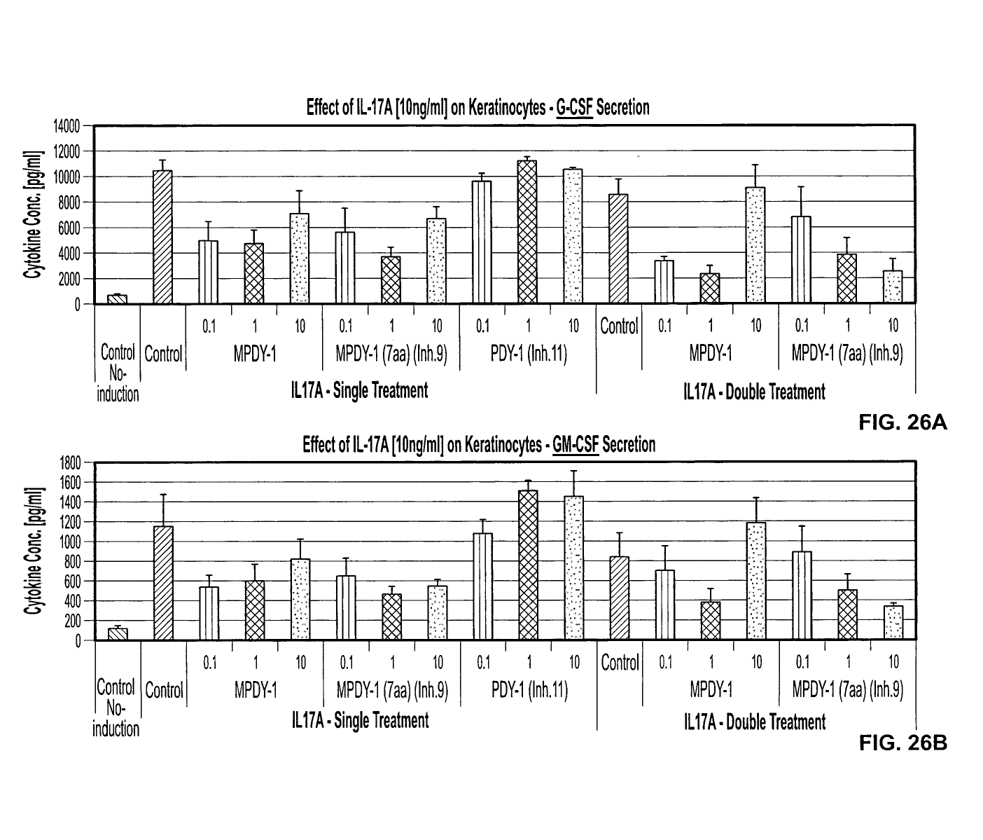

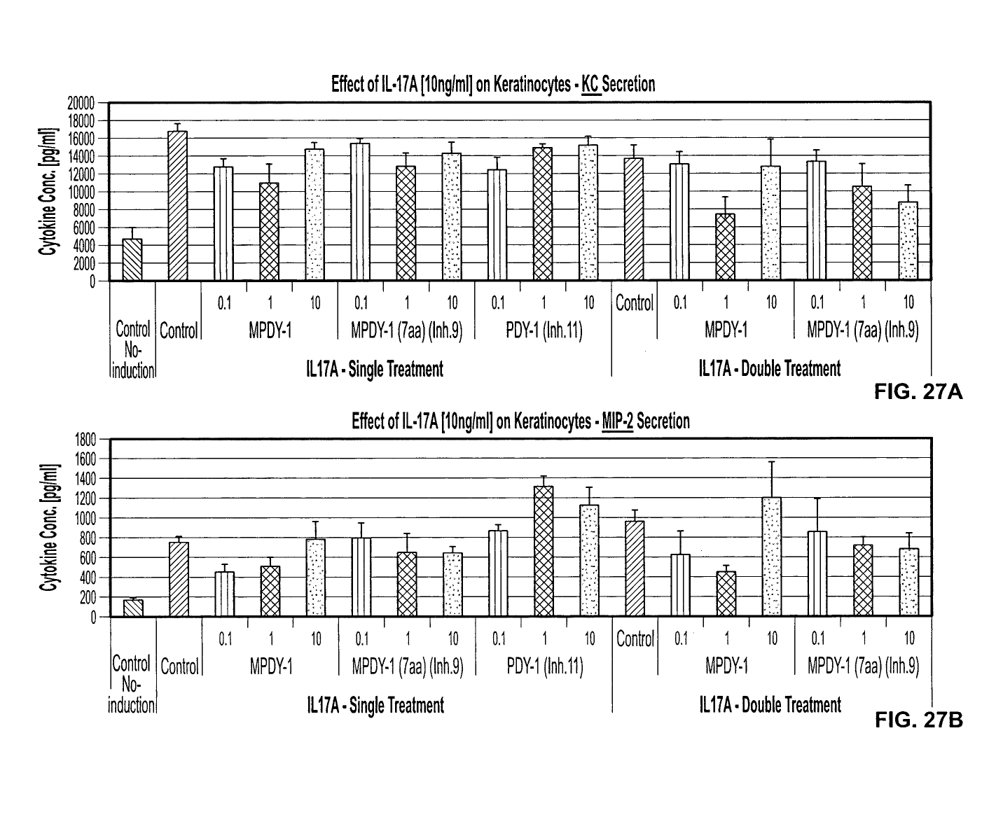

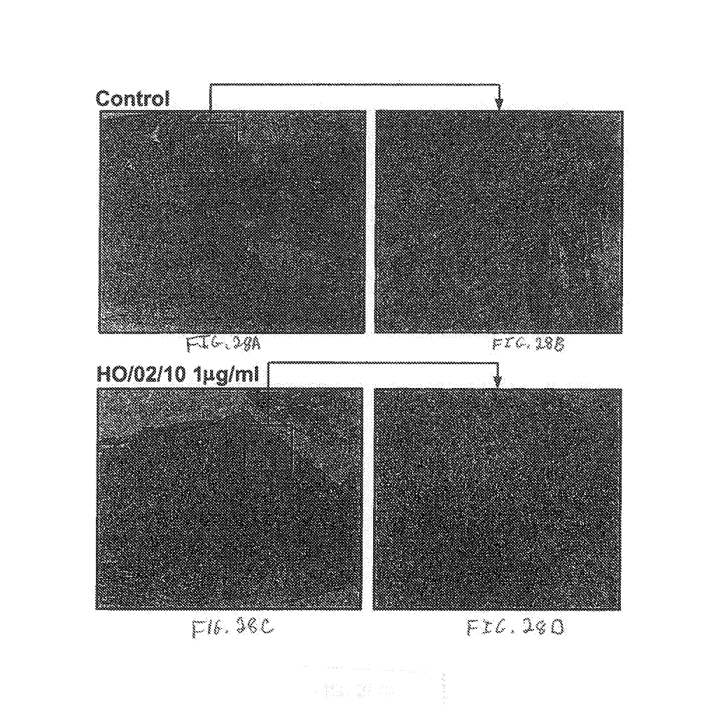

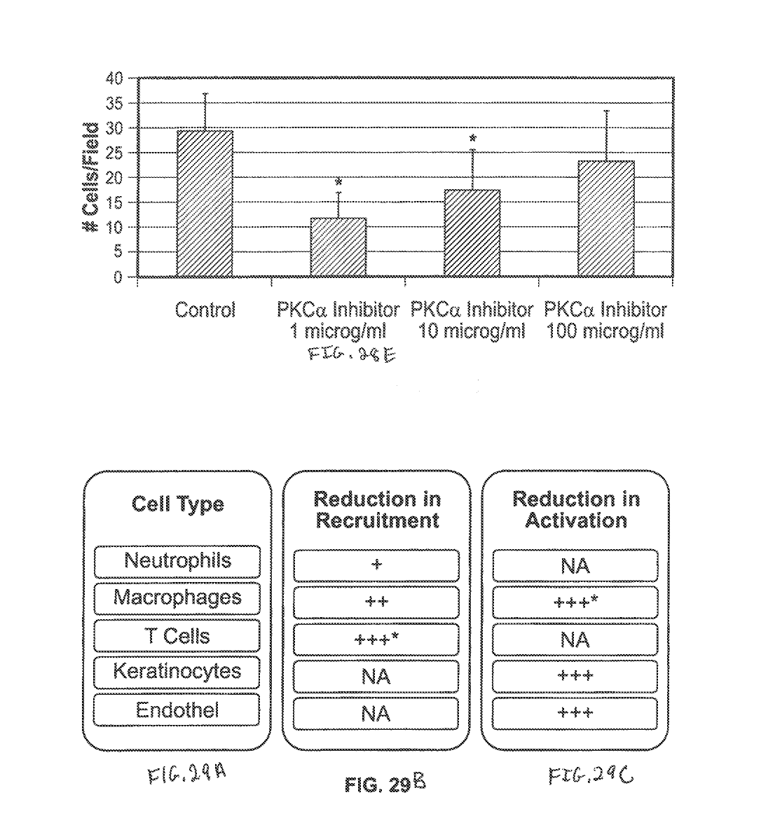

FIG. 11, as well as FIGS. 17-27 demonstrate the ability of HO/02/10 to decrease dramatically the secretion of major pro-inflammatory cytokines, such as TNFα, IL-1 and IL-6. Specifically, IL-6 was shown to be essential for the development of TH17 cells that are involved in the pathogenesis of psoriasis, with enhancing effect demonstrated for IL-1 and TNFα. TNFα and IL-6 are known targets for psoriasis therapy. FIG. 11 demonstrates the effect of 1 μg/ml HO/02/10.

HO/02/10 was also shown to inhibit basal keratinocyte and endothelial cell immunological activation in vivo. ICAM is an adhesion molecule that allows leukocytes infiltration into inflammatory lesions. Specifically in skin, basal keratinocytes express ICAM-1 upon immunological activation which may enhance infiltration of neutrophils and CD8-T cells into the epidermis, one of the hallmarks of psoriasis. Thus, the effect of HO/02/10 on ICAM expression in skin was examined by immunohistochemistry in a wound inflammatory setting in vivo.

Down regulation of activated keratinocytes and endothelial cells (ICAM-1 staining) in skin inflammation was observed. A two-cm longitudinal incision was done on the upper back of a C57BL/6J mouse, Following wounding, a sterile pad was sutured to the mouse's skin. Animals were treated daily with HO/02/10 (n=12). Five days post-wounding, when inflammatory phase reaches its peak, the mice were sacrificed, skin tissues were embedded in paraffin and immunohistochemical staining was performed utilizing anti-ICAM-1 antibodies.

As shown in FIGS. 12A-12F, HO/02/10 dramatically reduces ICAM expression on basal keratinocytes and endothelial in blood vessels of the skin. This effect was shown to be dose dependent with maximal effect, demonstrated at 10 μg/ml.

FIGS. 13A-13D show additional stains showing down regulation of activated keratinocytes and endothelial cells (ICAM-1 staining) in skin inflammation. As above, a two-cm longitudinal incision was done on the upper back of a C57BL/6J mouse. Following wounding, a sterile pad was sutured to the mouse's skin. Animals were treated daily with MPDY-1 (n=6). Five days post-wounding, when inflammatory phase reaches its peak, the mice were sacrificed, skin tissues were embedded in paraffin and immunohistochemical staining was performed utilizing anti-ICAM-1 antibodies.

FIG. 14 is a histogram comparing the percent of mice exhibiting positive ICAM-1 staining at both wound edges.

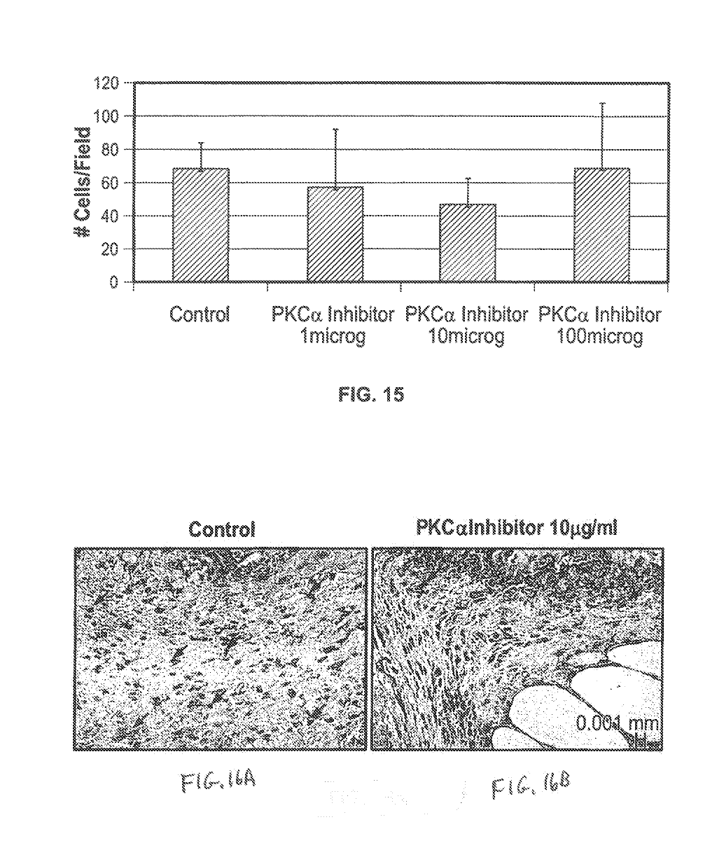

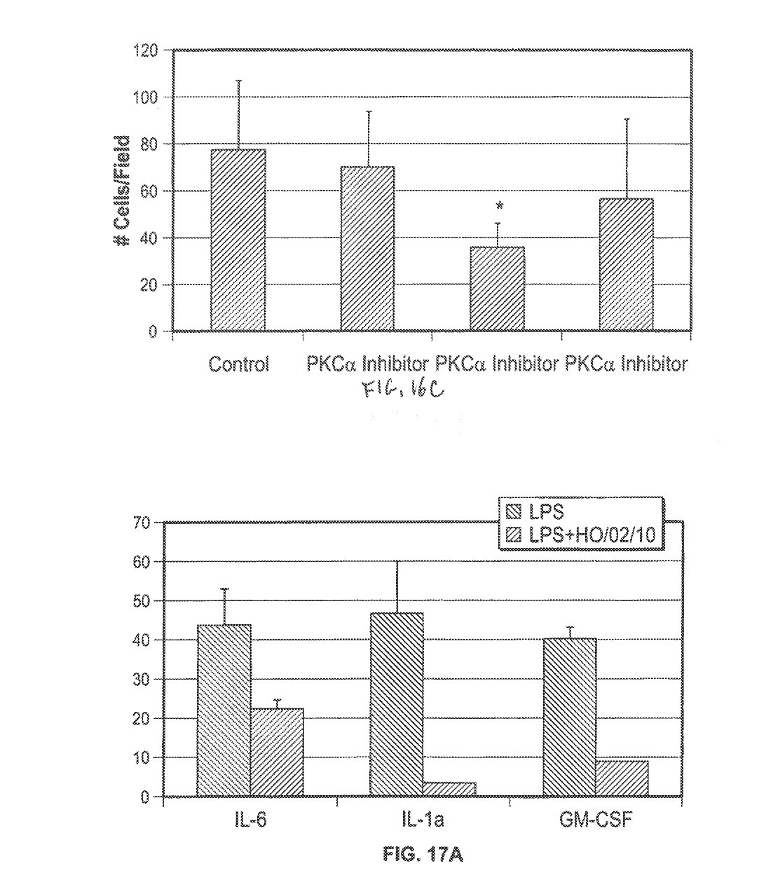

The effect of MPDY-1 on macrophage infiltration was also shown by Iba-1 staining. Iba-1 is a general marker for macrophages. FIG. 15 is a histogram showing comparing the number of cells per field exhibiting positive Iba-1 staining. As above, a two-cm longitudinal incision was done on the upper back of a C57BL/6J mouse. Following wounding, a sterile pad was sutured to the mouse's skin. Animals were treated daily with MPDY-1 (n=6). Five days post-wounding, when inflammatory phase reaches its peak, the mice were sacrificed, skin tissues were embedded in paraffin and immunohistochemical staining was performed utilizing anti-Iba-1 antibodies. A dose dependent effect of MPDY-1 on macrophage infiltration was observed.

The effect of MPDY-1 on macrophage activation was also shown by MAC-2 staining. MAC-2 is a specific marker for activated macrophages. FIGS. 16A-16C show a series of MAC-2 stains and a histogram comparing the number of cells per field exhibiting positive MAC-2 staining. A two-cm longitudinal incision was done as described above. Animals were treated daily with DPBS^{-/-} (Control) or MPDY-1 in the specified concentrations (n=6). After 5 days immunohistochemical staining was performed utilizing anti-MAC-2 antibodies. Bar 1 μm. (*p(control Vs. MPDY-1 10 μg)=0.0028). Activation of macrophages was significantly inhibited following MPDY-1 treatment.

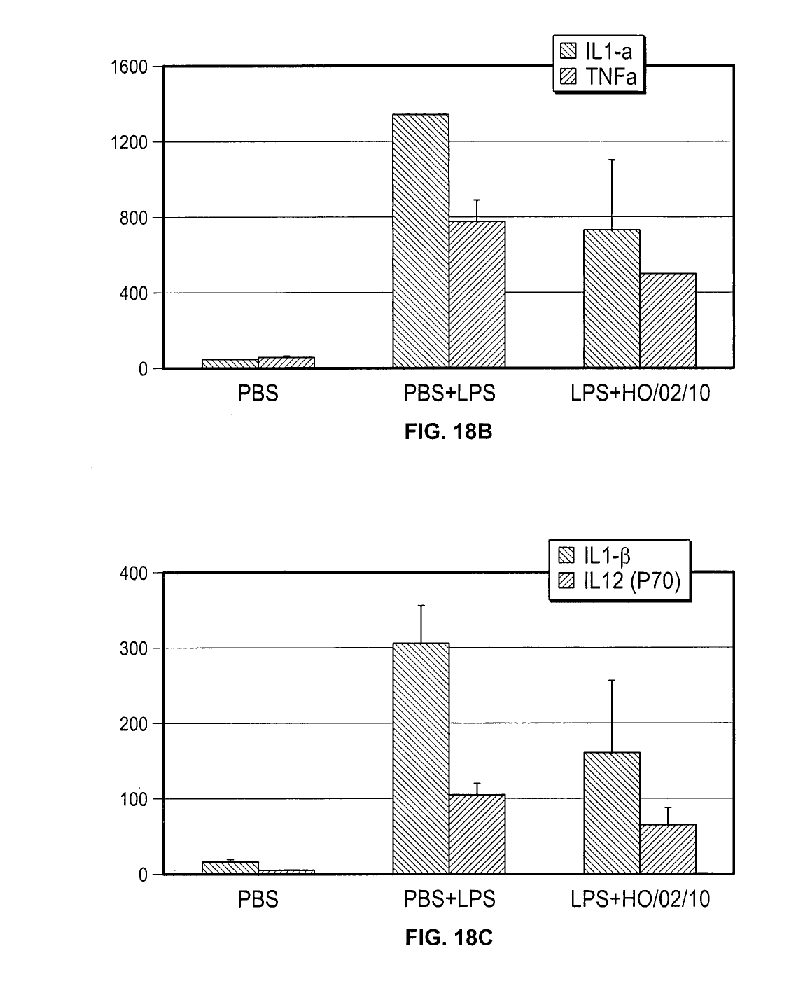

HO/02/10 was also shown to decrease cytokine secretion from activated keratinocytes and macrophages. In recent years it was found that both immune and skin components are equally contributing to the cycle underlying psoriatic pathogenesis. Resident skin cells and immunological cells (both resident and infiltrating cells) interact in the inflammatory psoriatic process by cell-cell interactions and cytokine secretion. Thus, HO/02/10 was examined for its direct effect on the secretion of pro-inflammatory, chemoattractant and immunological pathway related cytokines form both keratinocytes and immune cells such as macrophages and dendritic cells. The results depicted in FIGS. 17 and 18 demonstrate that HO/02/10 down regulates secretion of immune related cytokines such as IL-6, IL-1α, GM-CSF, MIP-2 and KC from keratinocytes and macrophages.

The results of FIGS. 17A and 17B show the effect of HO/02/10 on cytokine secretion in keratinocytes. Keratinocytes were derived from newborn C57BL/6 mice skin. The cells were incubated for 5 days in 24 wells plates. Cells were then treated with DPBS−/−, LPS (100 ng/ml), or HO/02/10 (1 μg/ml)+LPS (100 mg/ml). Medium containing secreted cytokines was collected after 48 hr and analyzed using a Luminex system.

The results of FIG. 18 show that HO/02/10 down regulates cytokine secretion in macrophages. Bone marrow cells were derived from B6 mice. Cells were incubated for 6 days in the presence of GM-CSF (20 ng/ml), and then were treated with DPBS−/−, LPS (100 ng/ml) or HO/02/10+LPS (1 μg/ml and 100 ng/ml, respectively).

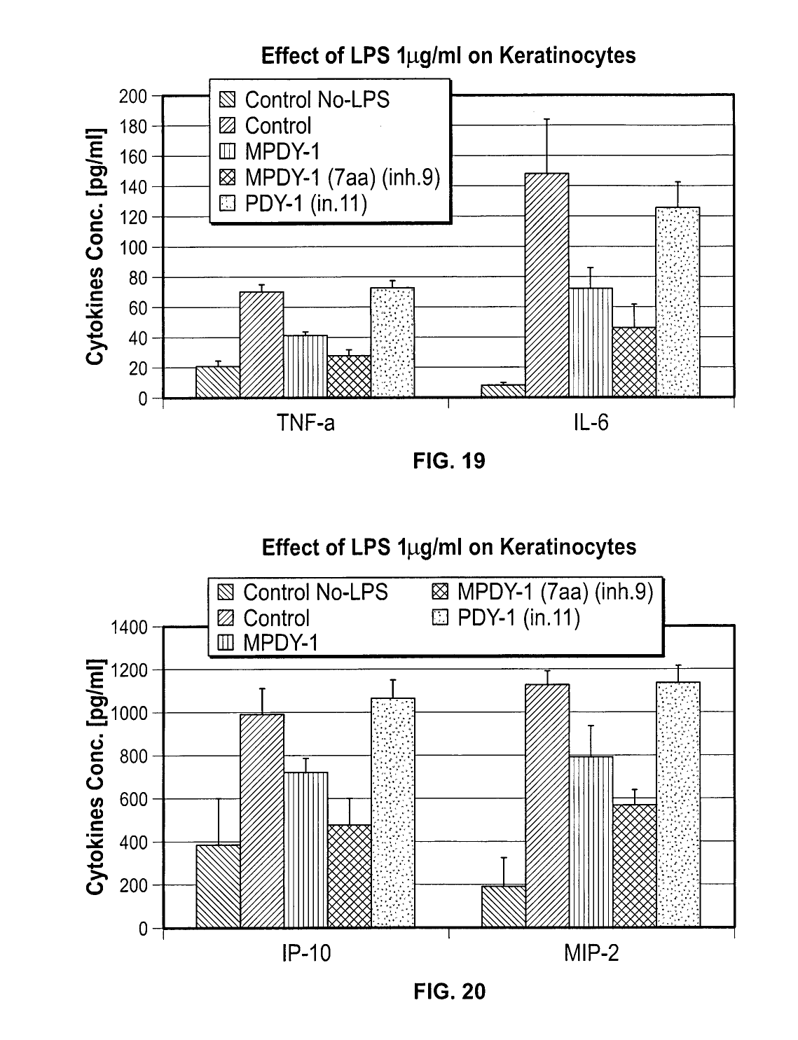

Other peptide PKCα inhibitors were also shown to decrease cytokine secretion from activated keratinocytes and macrophages. FIGS. 19 to 23 show that the peptide inhibitors MPDY-1 (SEQ ID NO: 6), MPDY-1 sh (SEQ ID NO: 12) and PDY-1 (SEQ ID NO: 13) decrease cytokine secretion from LPS and TNFα activated keratinocytes. FIGS. 24 to 27 show that the peptide inhibitors MPDY-1 (SEQ ID NO: 6), MPDY-1 sh (SEQ ID NO: 12) and PDY-1 (SEQ ID NO: 13) decrease cytokine secretion from IL-17A activated keratinocytes.

Table 3 summarizes the results according to cytokine roles and origin for HO/02/10.

TABLE 3
HO/02/10 Effect On Stimulated Mice Derived-Cells

| | | Chemo- | | | |
|---|---|---|---|---|---|
| | Pro- | attractants | Systemic | Th1 | Th17 |
| | inflammatory | (% | (% | (% | (% |
| | (% inhibition) | inhibition) | inhibition) | inhibition) | inhibition) |
| Keratinocytes | IL-1 (80%) | KC | GM-CSF | | IL-6 |
| | IL-6 (40%) | (65%) | (50%) | | (40%) |
| | | MIP-2 | G-CSF | | |
| | | (30%) | (30%) | | |
| Spleen | IL-1 (50%) | | | | |
| | IL-6 (40%) | | | | |
| | TNFa (50%) | | | | |
| Bone marrow | IL-1 50% | KC | G-CSF | IL-12 | |
| macrophages | TNFa (50%) | (40%) | (40%) | (40%) | |
| | | MIP-2 | | TNFα | |
| | | (30%) | | (50%) | |
| Bone marrow | IL-6 (30%) | | | IP-10 | |
| DCs | | | | (20%) | |

HO/02/10 was also shown to attenuate T cells infiltration to the skin. The effect of HO/02/10 on T cell infiltration was studied in viva using anti-CD3 specific staining.

As can be seen in FIGS. 28A-28D, HO/02/10 down regulated T cell infiltration to the dermis and epidermis during the inflammatory stage. Specifically HO/02/10 inhibited T cell infiltration into the epidermis which indicates additional anti-inflammatory properties also characteristic of psoriasis plaques. A two-cm longitudinal incision was done as described above. Animals were treated daily with HO/02/10 (n=12), After nine days immunohistochemical staining was performed utilizing anti-CD3 antibodies. FIG. 28B is a histogram comparing the number of cells per field positively stained for CD3. The effect was statistically significant at concentrations of 1 μg/ml and 10 μg/ml, where 1 μg/ml treatment demonstrates stronger effects than 10 μg/ml.

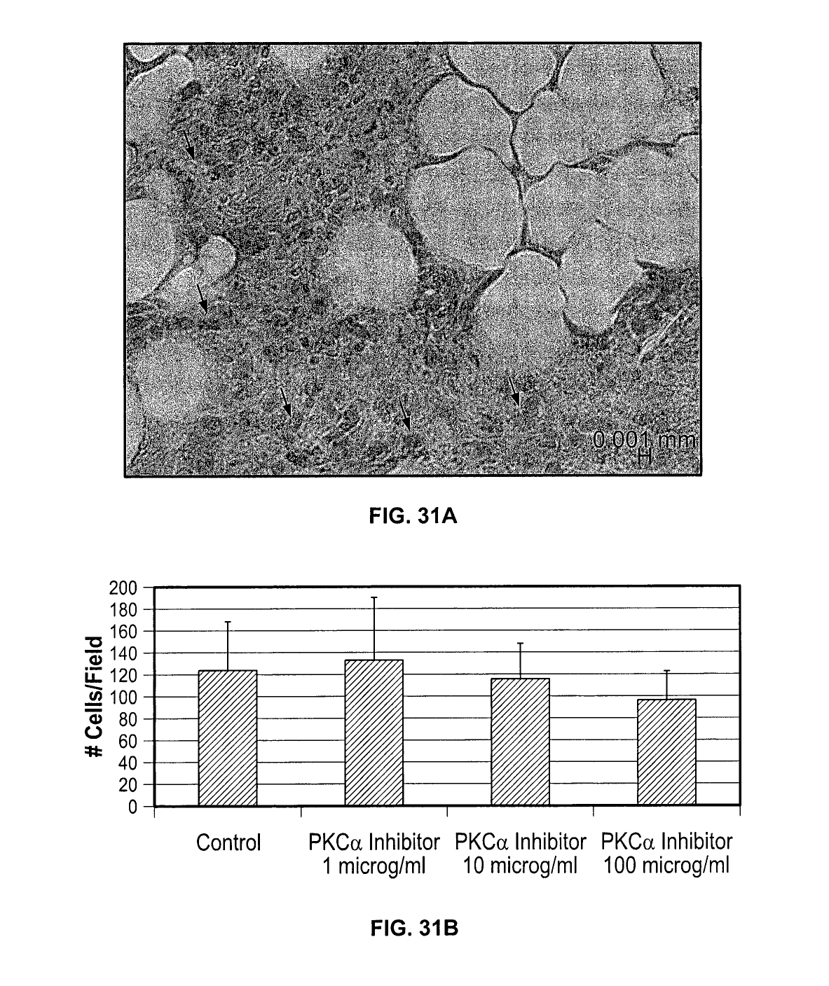

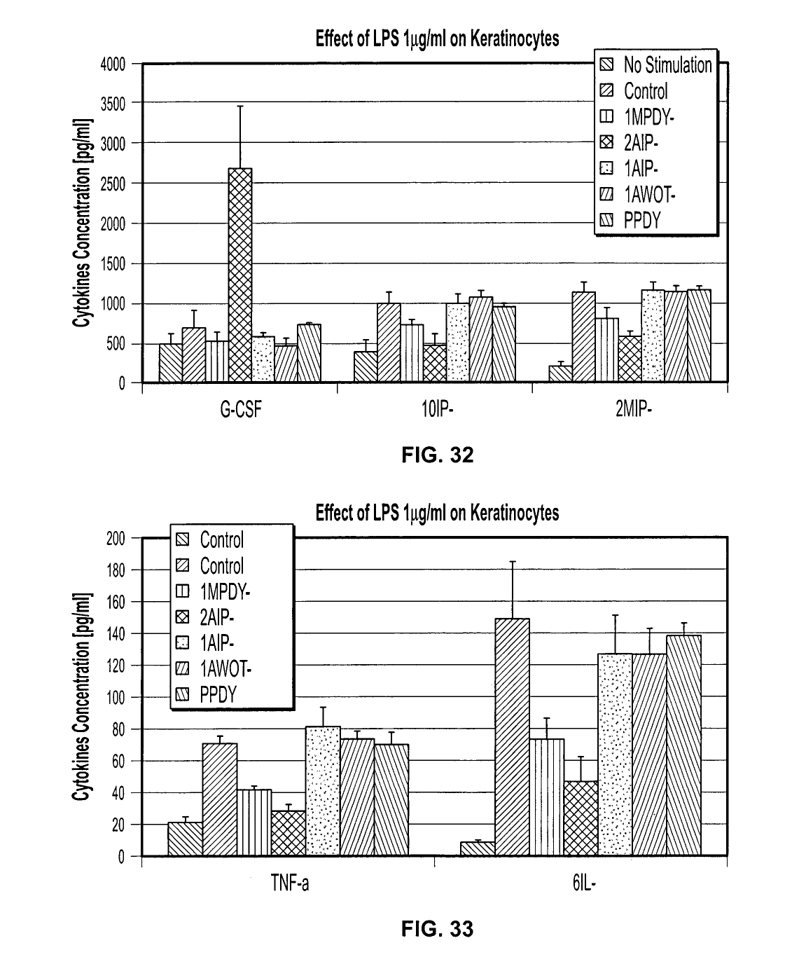

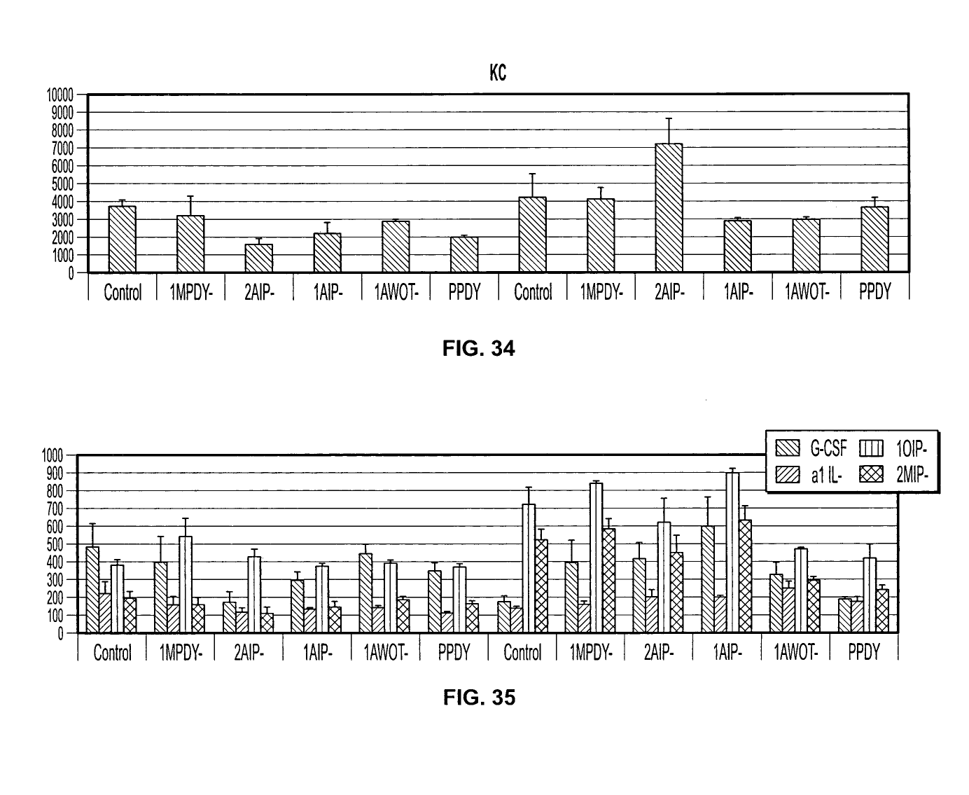

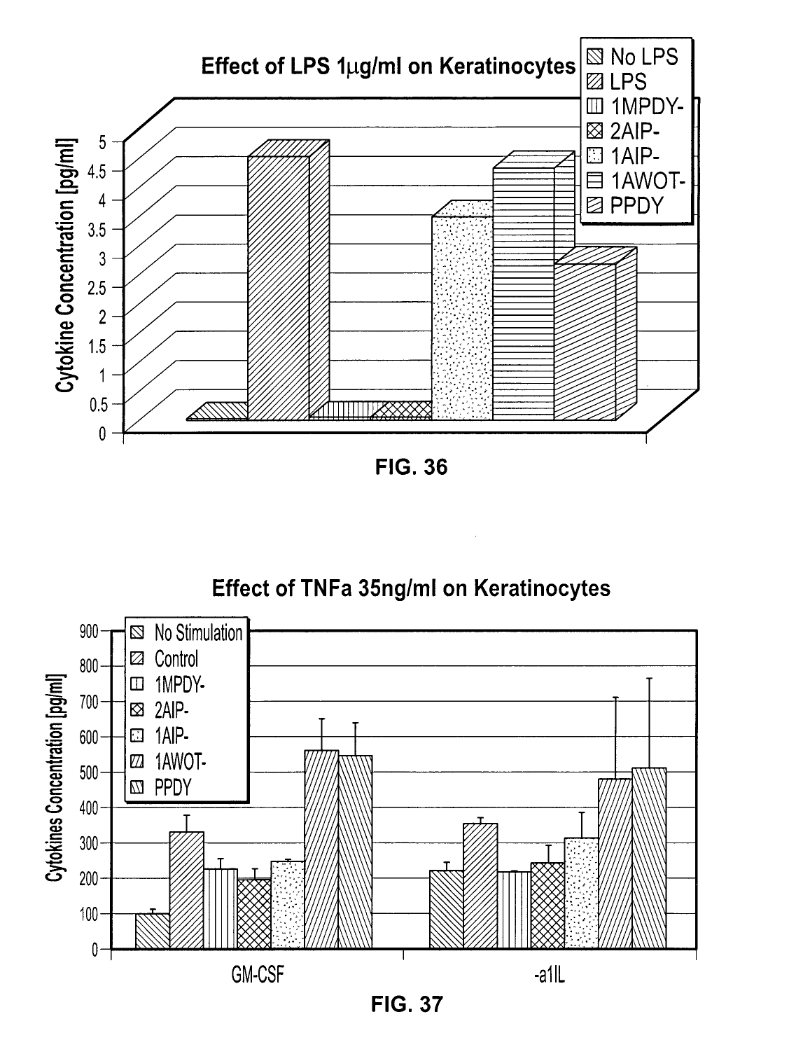

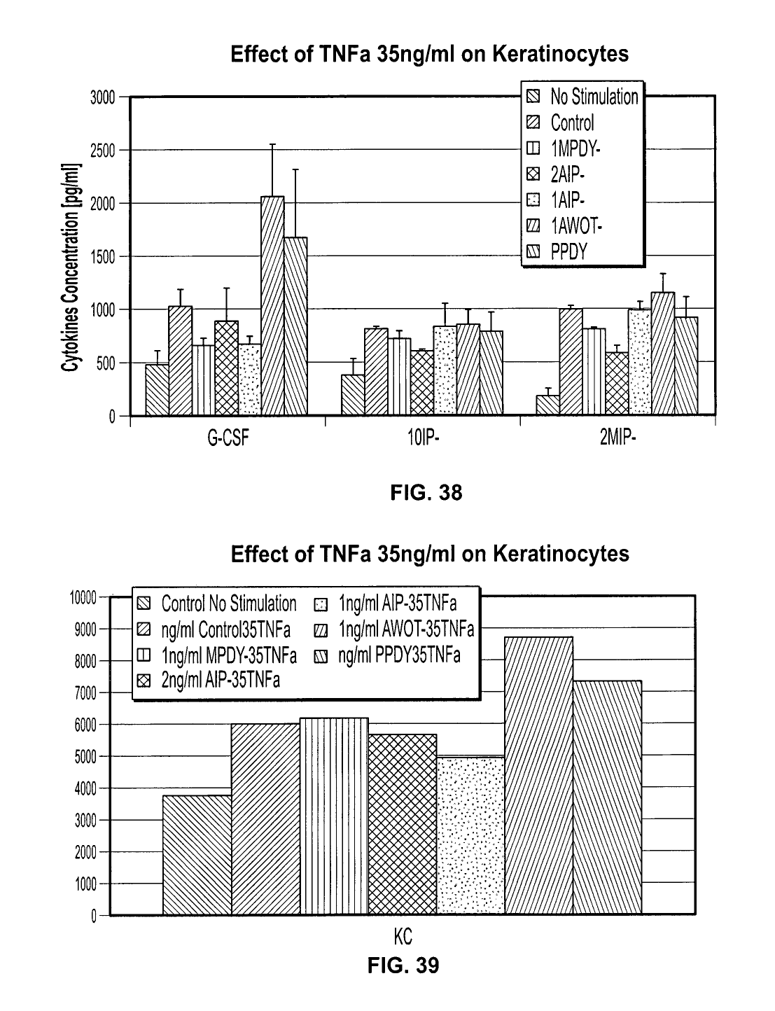

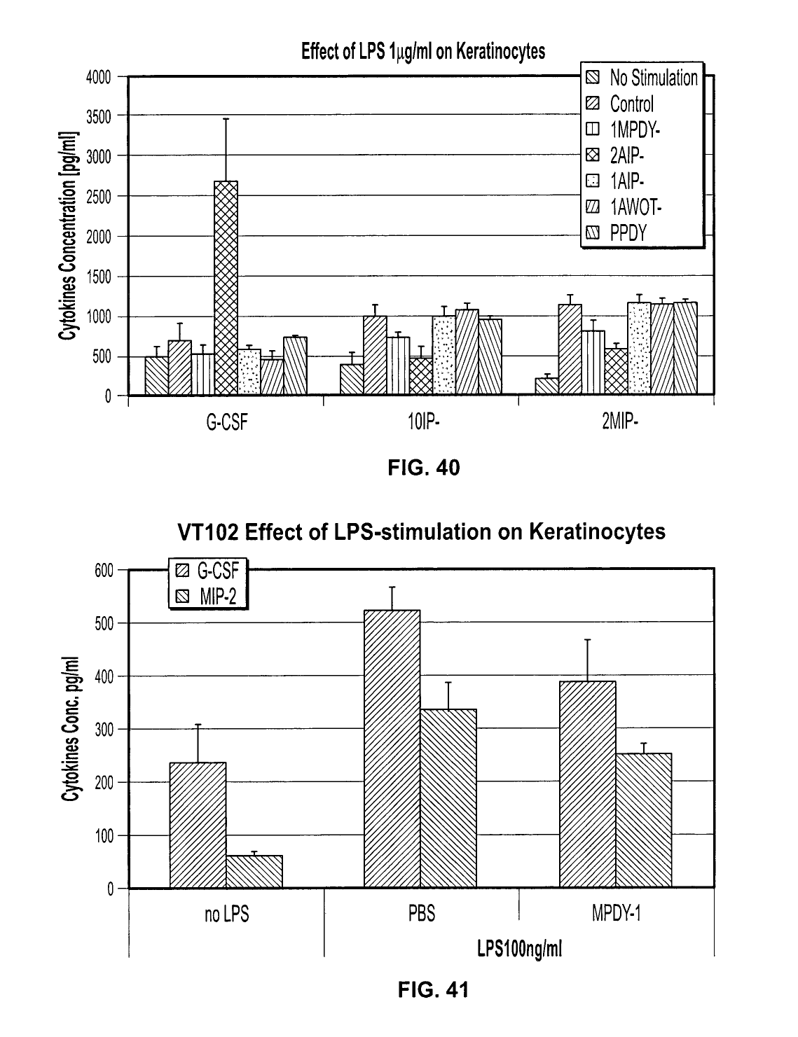

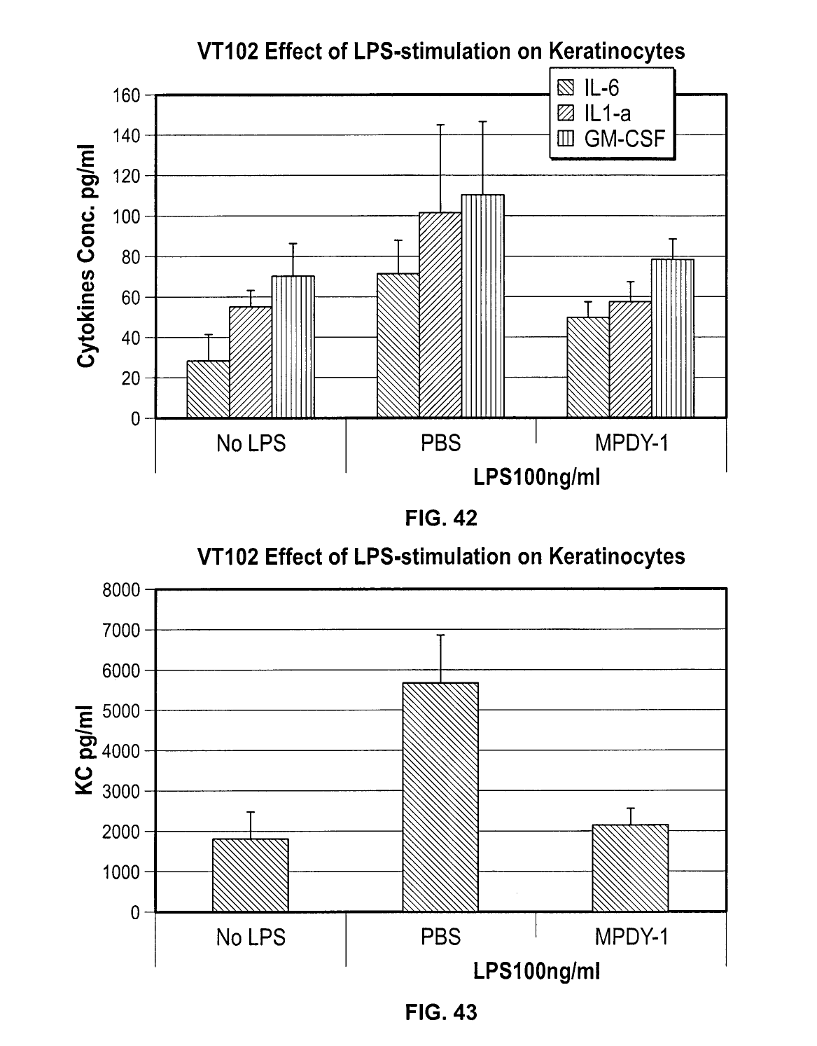

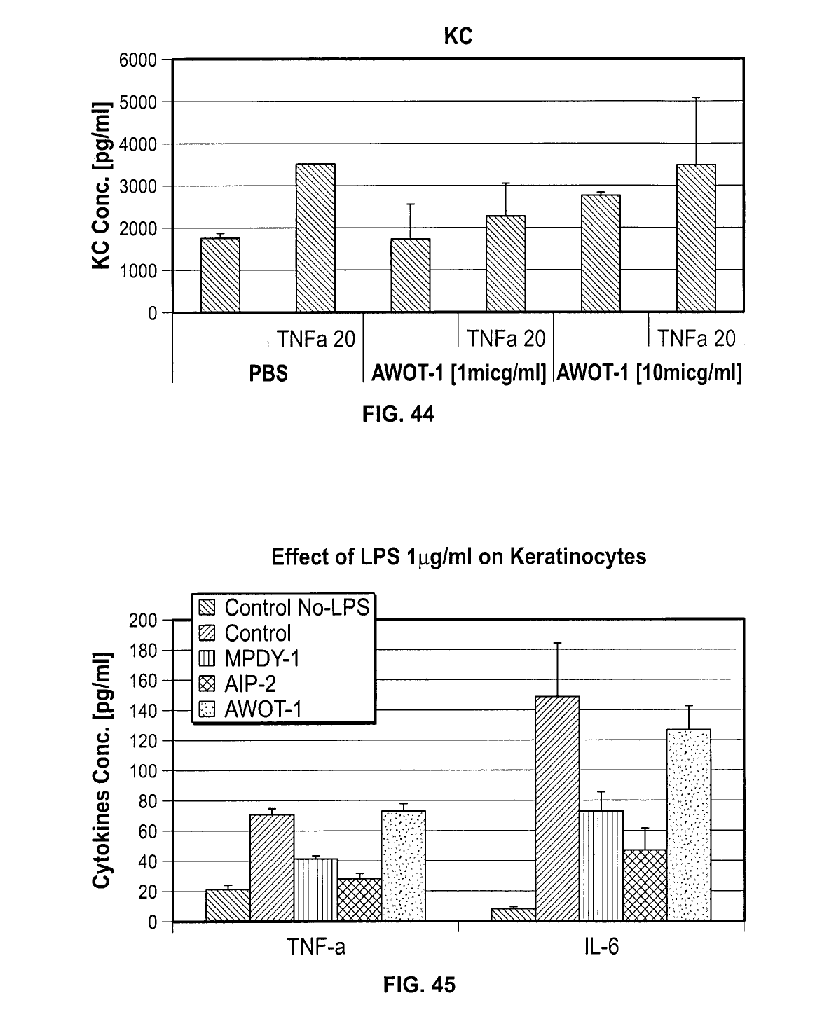

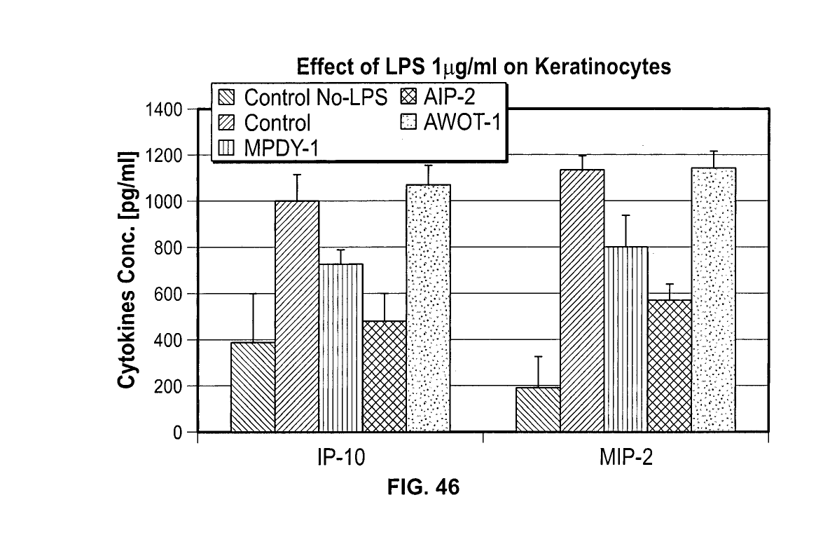

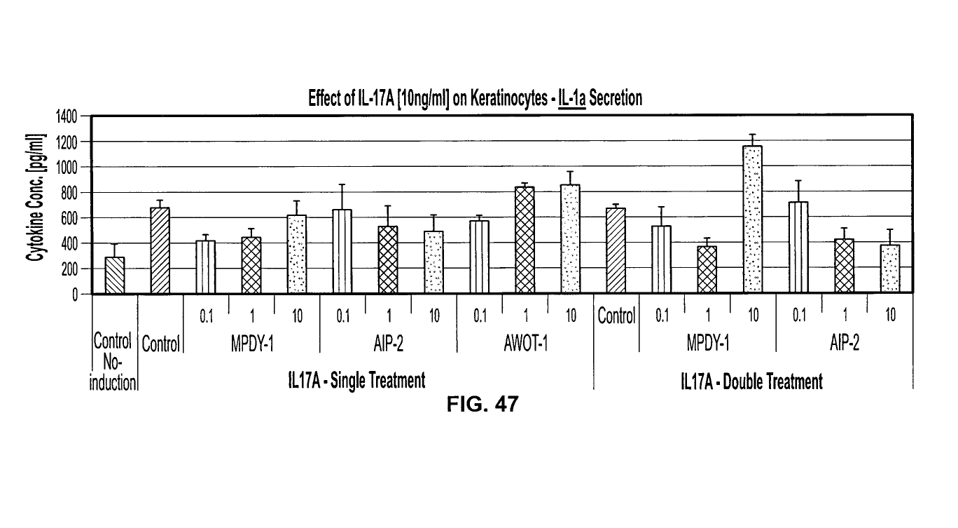

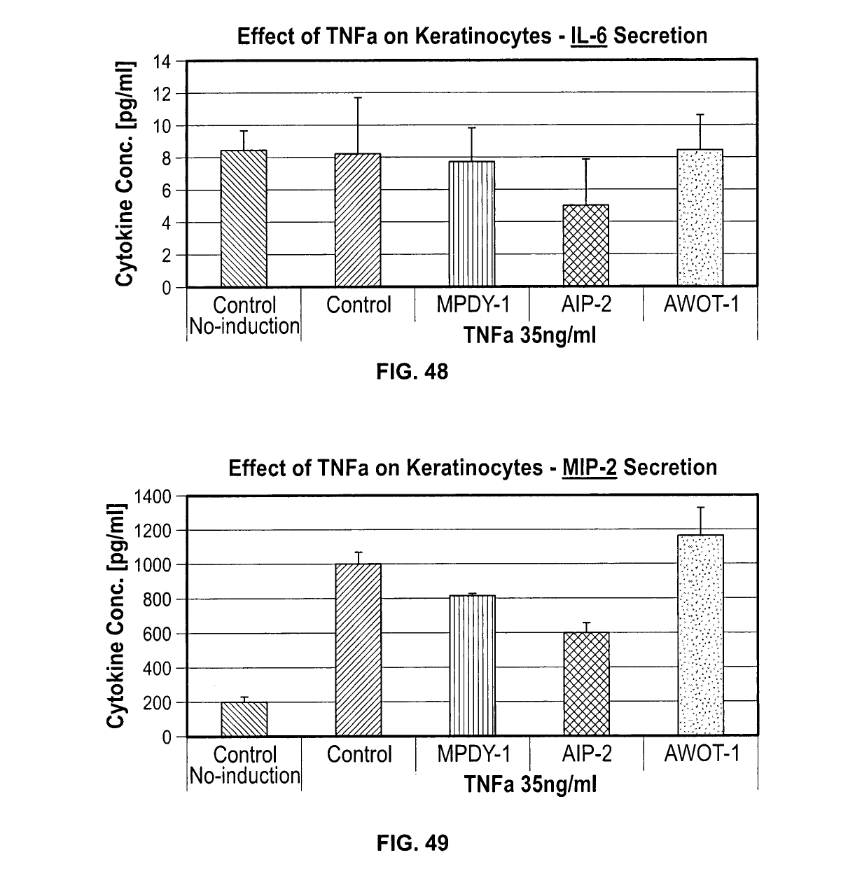

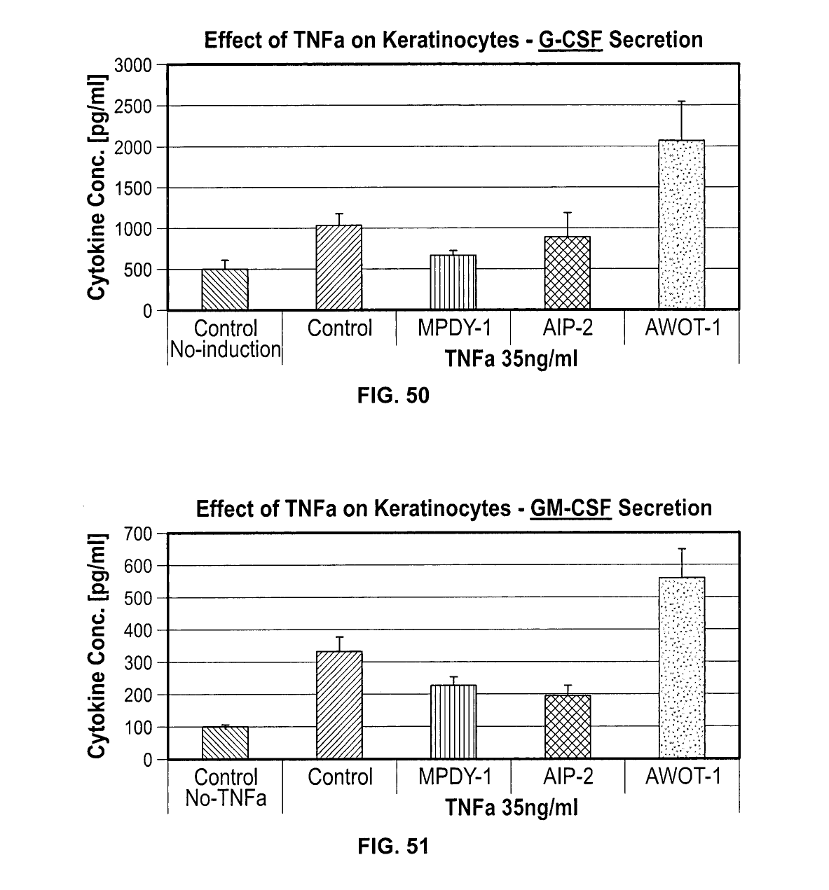

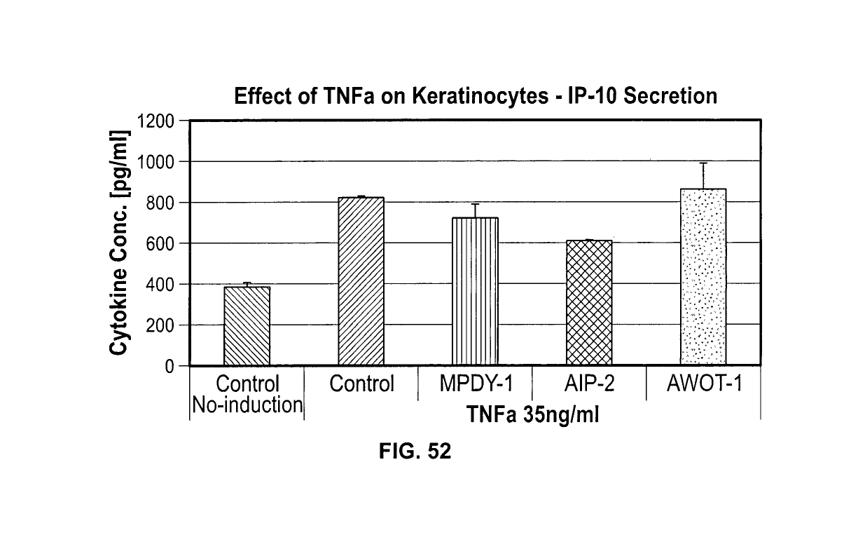

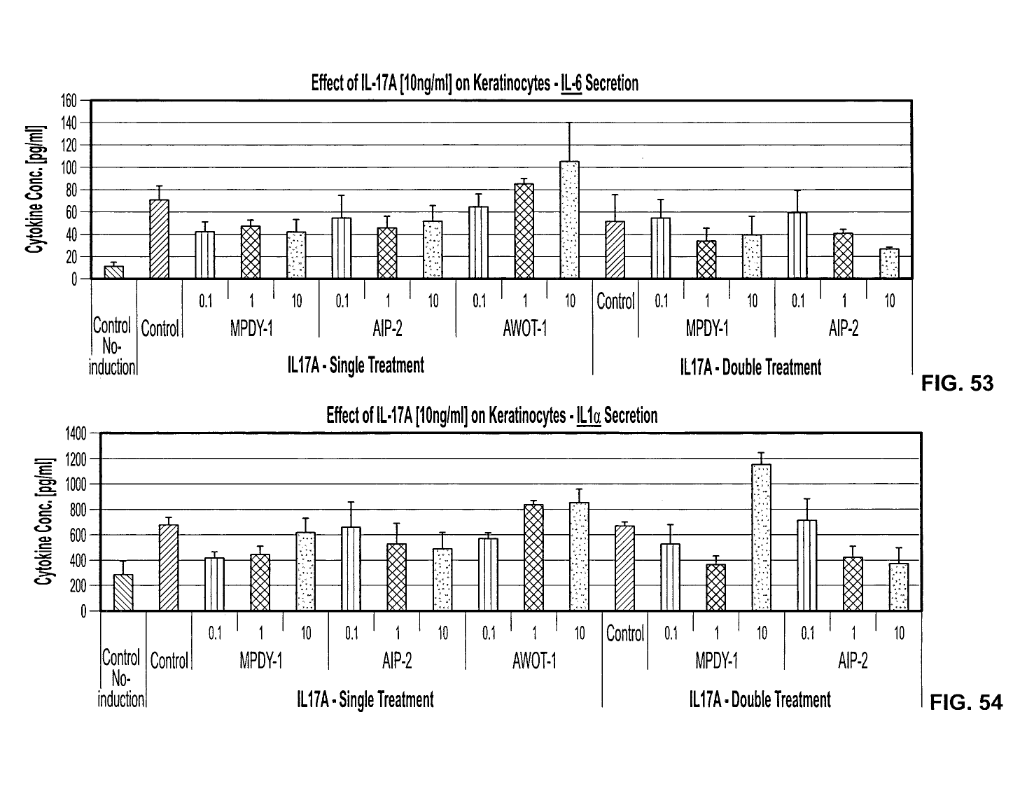

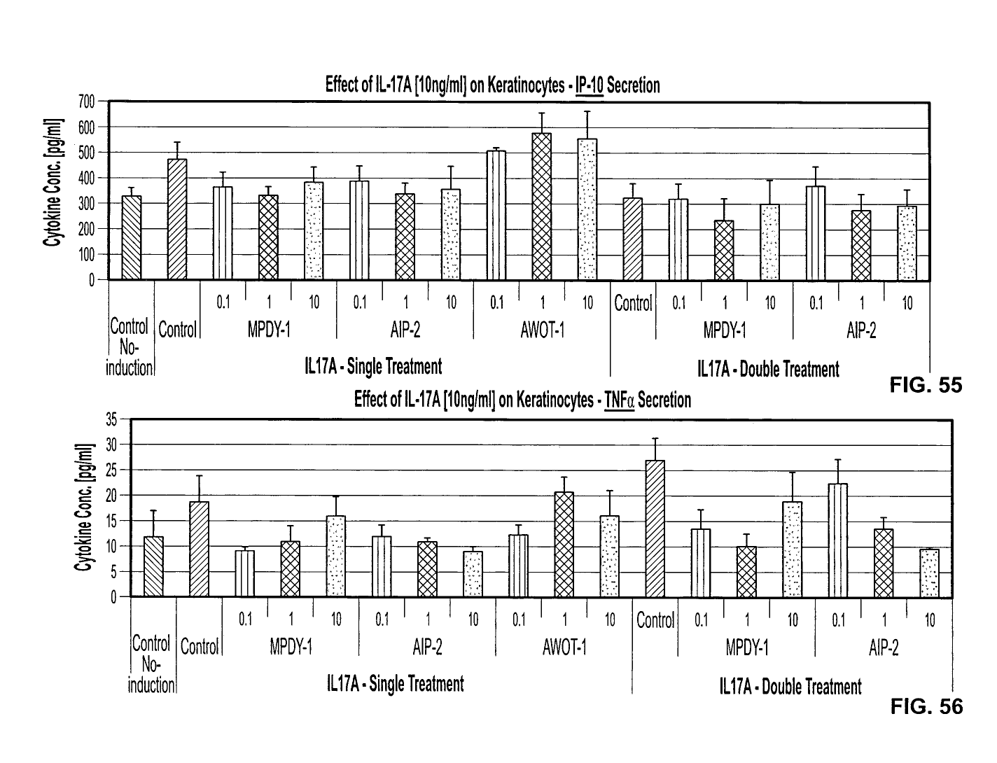

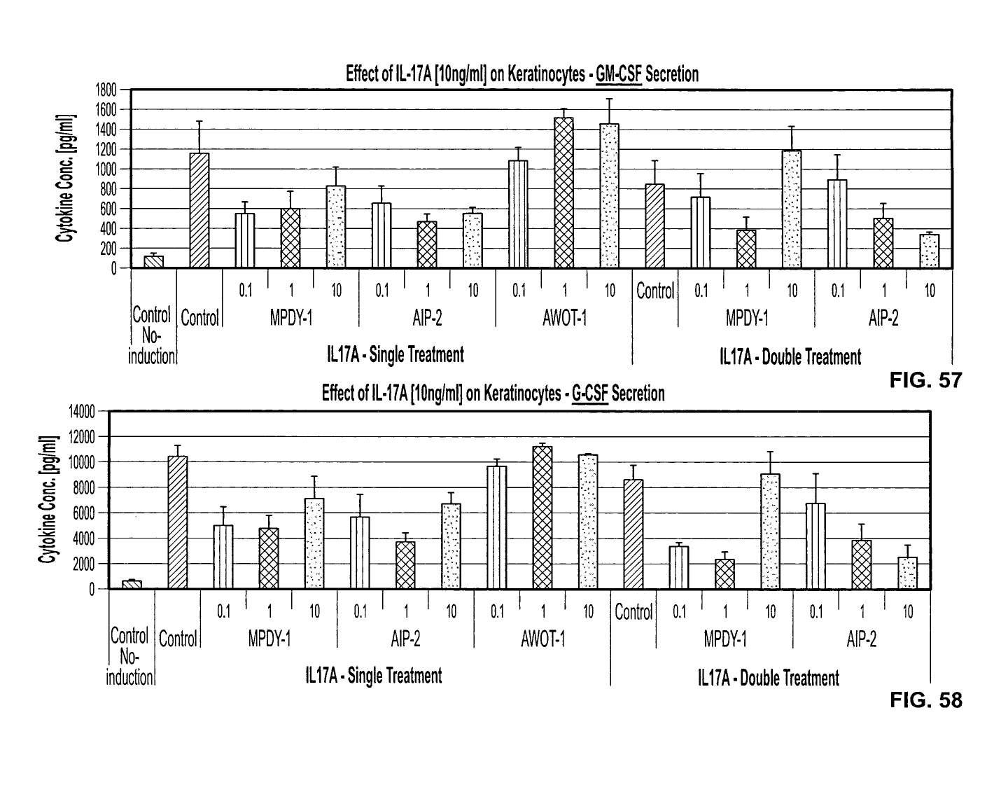

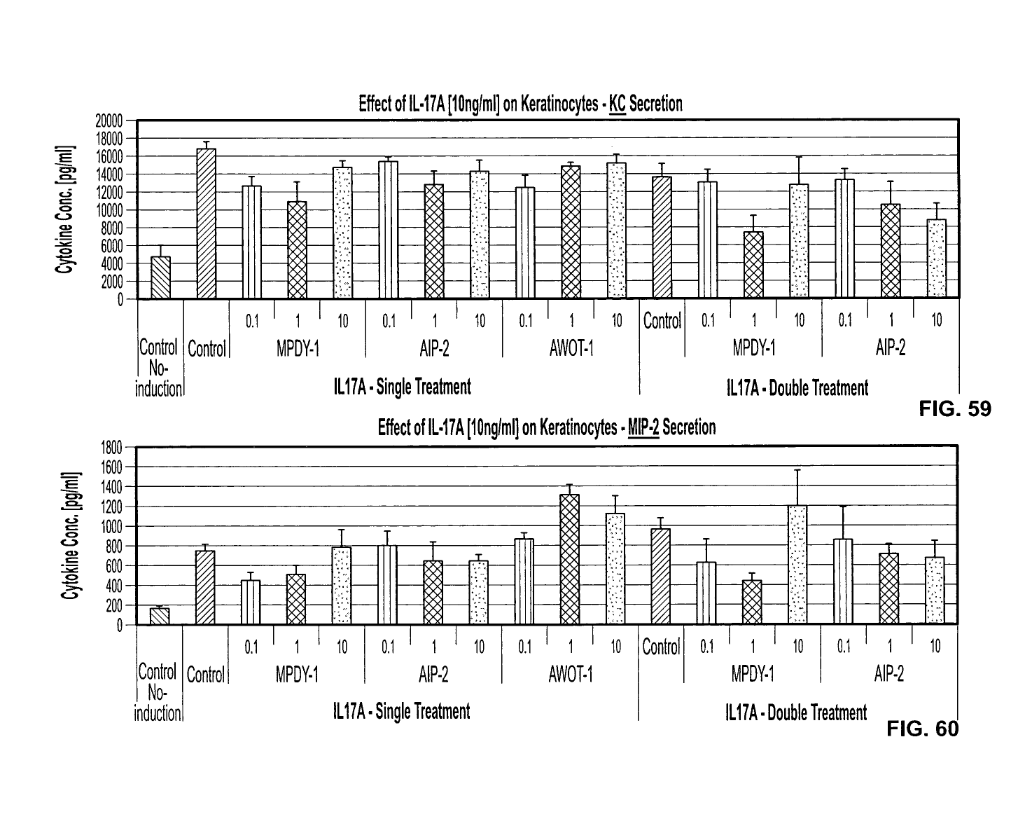

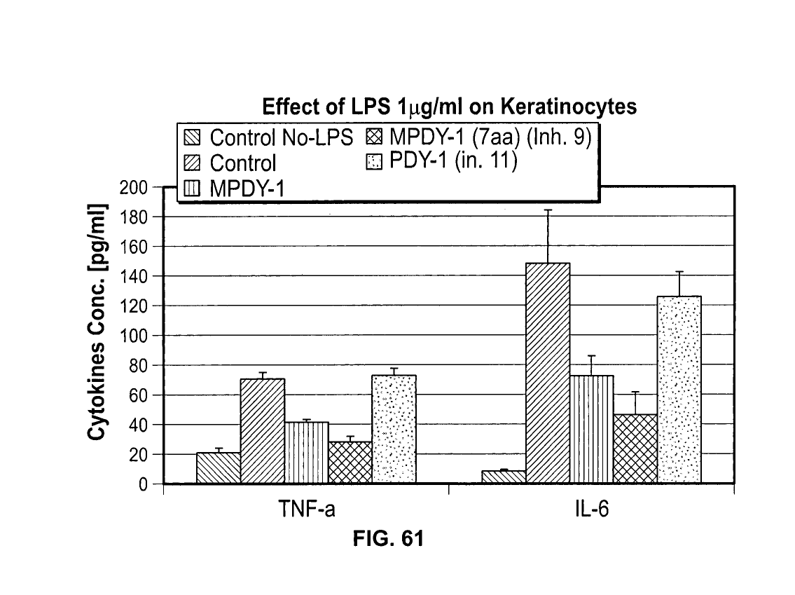

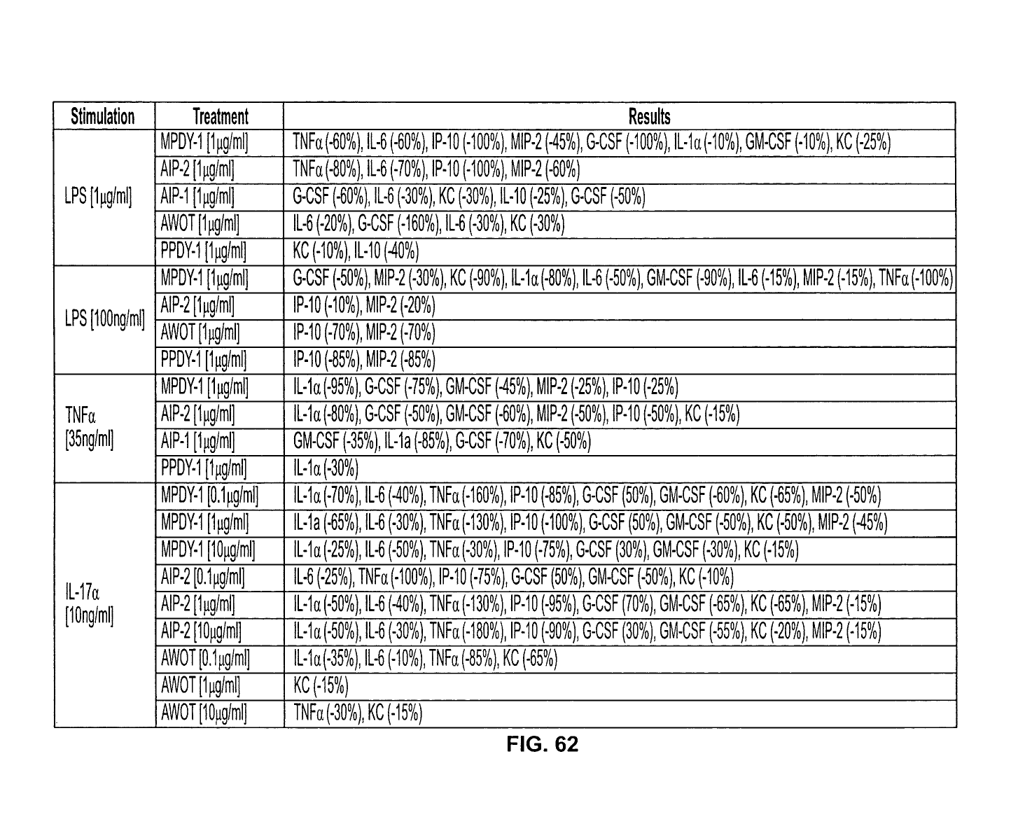

HO/02/10 was also shown to attenuate neutrophil infiltration to the skin (FIG. 31). The effect of HO/02/10 on neutrophil infiltration was studied in vivo using neutrophil specific staining. A two-cm longitudinal incision was done as described above. Animals were treated daily with DPBS−/− (Control) or PKCα inhibitor in the specified concentrations (n=6). After five days the mice were sacrificed, skin tissues were embedded in paraffin and immunohistochemical staining for neutrophils was performed. Although a dose dependent trend was observed, results were not statistically significant.

FIGS. 29A-29C presents a summary of the effects of HO/02/10 on different cell types.

In summary, the mechanism of action of PKCα inhibitors was determined implicating their use as an effective therapy for psoriasis. PKCα inhibitors were shown to 1) normalize epidermal differentiation markers expression by reducing terminal differentiation; 2) attenuate abnormal hyper-proliferation; 3) regulate skin structure and augment skin strength; and 4) down-regulate inflammation by differentially affecting different cell type recruitment and activation in various steps of the inflammatory process.

FIG. 30 shows a schema depicting the overall effect of the PKCα inhibitors of the present disclosure on the psoriatic related pathway. The scheme summarizes the inhibitory effect of the inhibitors on various cell types and inflammatory stages in the skin. PKCα inhibitors inhibit secretion of pro-inflammatory cytokines (such as, IL-1, IL-6 and TNFα) by resident skin immune cells. Accordingly, a decrease in endothelial cells and keratinocytes activation is achieved, resulting a significant reduction in ICAM-1 expression, chemokines secretion and reduce in leukocytes infiltration to the site of inflammation, including neutrophils, macrophages, and T-cells. Cytokines involved in the development and progression of the Th1 and Th17 pathways, both main pathways in psoriasis, were also down regulated.

Although the objects of the disclosure have been described with reference to the above example, it will be understood that modifications and variations are encompassed within the spirit and scope of the disclosure. Accordingly, the disclosure is limited only by the following claims.

1. A method of treating psoriasis in a subject comprising, administering to the subject an inhibitor of PKCα, thereby treating psoriasis in the subject.

2. The method of claim 1, wherein the inhibitor of PKCα is a polypeptide.

3. The method of claim 2, wherein the polypeptide comprises an amino acid sequence selected from SEQ ID NOs: 1-5.

4. The method of claim 3, wherein the polypeptide comprises an N-terminal modification, C-terminal modification, or combination thereof.

5. The method of claim 2, wherein the polypeptide is selected from SEQ ID NOs: 1-5 and physiologically acceptable salts thereof.

6. The method of claim 5, wherein the polypeptide comprises an N-terminal modification, C-terminal modification, or combination thereof.

7. The method of claim 6, wherein the polypeptide is N-acylated.

8. The method of claim 7, wherein the polypeptide is N-myristoylated or N-palmitoylated.

9. The method of claim 2, wherein the polypeptide is selected from SEQ ID NOs: 6-13.

10. The method of claim 2, wherein the polypeptide is formulated for topical administration and is administered topically.

11. The method of claim 10, wherein the polypeptide is formulated as a gel, ointment, cream, foam or spray.

12. The method of claim 10, wherein the polypeptide is formulated as a cream.

13. The method of claim 10, wherein the polypeptide is administered at a dose of about 0.1 to about 1000 micrograms per kilogram.

14. The method of claim 13, wherein the polypeptide is administered at a dose of about 1.0 to about 50 micrograms per kilogram.

15. The method of claim 14, wherein the polypeptide is administered daily, weekly, biweekly or monthly.

16. The method of claim 1, wherein the psoriasis is plaque psoriasis.

17. A kit for treating psoriasis in a subject comprising:
a) an inhibitor of PKCα; and
b) instructions for administering the inhibitor to the subject.

18. The kit of claim 17, wherein the inhibitor of PKCα is a polypeptide.

19. The kit of claim 18, wherein the polypeptide comprises an amino acid sequence selected from SEQ ID NOs: 1-5.

20. The kit of claim 19, wherein the polypeptide comprises an N-terminal modification, C-terminal modification, or combination thereof.

21. The kit of claim 18, wherein the polypeptide is selected from SEQ ID NOs: 1-5 and physiologically acceptable salts thereof.

22. The kit of claim 21, wherein the polypeptide comprises an N-terminal modification, C-terminal modification, or combination thereof.

23. The kit of claim 22, wherein the polypeptide is N-acylated.

24. The kit of claim 23, wherein the polypeptide is N-myristoylated or N-palmitoylated.

25. The kit of claim 18, wherein the polypeptide is selected from SEQ ID NOs: 6-13.

26. The kit of claim 18, wherein the polypeptide is formulated for topical administration and is administered topically.

27. The kit of claim 26, wherein the polypeptide is in a form selected from the group consisting of a gel, an ointment, a cream, a foam and a spray.

28. The kit of claim 27, wherein the polypeptide is formulated as a cream.

29. The kit of claim 26, wherein the instructions specify that the polypeptide is administered at a dose of about 0.1 to about 1000 micrograms per kilogram.

30. The kit of claim 29, wherein the instructions specify that the polypeptide is administered at a dose of about 1.0 to about 50 micrograms per kilogram.

31. The kit of claim 26, wherein the wherein the instructions specify that the polypeptide is administered daily, weekly, biweekly or monthly.

32. The kit of claim 17, wherein the psoriasis is plaque psoriasis.

33-48. (canceled)


SEQUENCE LISTING (13 sequences)

SEQ ID NO: 1 - protein, 6 aa, Artificial Sequence
  Synthetic peptide
FARKGA

SEQ ID NO: 2 - protein, 9 aa, Artificial Sequence
  Synthetic peptide
FARKGALRQ

SEQ ID NO: 3 - protein, 7 aa, Artificial Sequence
  Synthetic peptide
FARKGAL

SEQ ID NO: 4 - protein, 8 aa, Artificial Sequence
  Synthetic peptide
FARKGARQ

SEQ ID NO: 5 - protein, 8 aa, Artificial Sequence
  Synthetic peptide
TLNPQWES

SEQ ID NO: 6 - protein, 9 aa, Artificial Sequence
  Synthetic peptide
FARKGALRQ

SEQ ID NO: 7 - protein, 9 aa, Artificial Sequence
  Synthetic peptide
FARKGALRQ

SEQ ID NO: 8 - protein, 7 aa, Artificial Sequence
  Synthetic peptide
FARKGAL

SEQ ID NO: 9 - protein, 8 aa, Artificial Sequence
  Synthetic peptide
TLNPQWES

SEQ ID NO: 10 - protein, 9 aa, Artificial Sequence
  Synthetic peptide
FARKGALRQ

SEQ ID NO: 11 - protein, 8 aa, Artificial Sequence
  Synthetic peptide
FARKGARQ

SEQ ID NO: 12 - protein, 7 aa, Artificial Sequence
  Synthetic peptide
FARKGAL

SEQ ID NO: 13 - protein, 9 aa, Artificial Sequence
  Synthetic peptide
FARKGALRQ